(19) United States
(12) Patent Application Publication
(10) Pub. No.: US 2022/0233537 A1
(43) Pub. Date: Jul. 28, 2022

(54) METHODS OF TREATING URINARY SYSTEM CANCERS

(71) Applicant: QED Therapeutics, Inc., San Francisco, CA (US)

(72) Inventors: Riccardo Panicucci, Bluffton, SC (US); Susan Arangio, Los Altos, CA (US); Craig Berman, Mill Valley, CA (US); Michael Monteith, Raleigh, NC (US); Harris Soifer, San Diego, CA (US); Gang Li, San Diego, CA (US); Carl Dambkowski, San Mateo, CA (US); Daniel Mulreany, San Francisco, CA (US)

(21) Appl. No.: 17/613,652

(22) PCT Filed: May 29, 2020
(86) PCT No.: PCT/US2020/035140

§ 371 Date: Nov. 23, 2021

Related U.S. Application Data
(60) Provisional application No. 62/906,194, filed on Sep. 26, 2019.
(60) Provisional application No. 62/855,277, filed on May 31, 2019.

(51) Int. Cl. A61K 31/506; C12Q 1/6886; C12Q 1/6874; G01N 33/574; A61K 31/7068; A61P 35/00; A61M 31/00

(52) U.S. Cl. CPC A61K 31/506; C12Q 1/6886; C12Q 1/6874; G01N 33/57492; A61K 31/7068; A61M 2210/1085; A61M 31/00; C12Q 2600/112; C12Q 2600/156; C12Q 2600/16; G01N 2800/52; A61P 35/00

(57) ABSTRACT

Provided herein are methods of treating an upper tract urothelial carcinoma in a patient by administering to the patient infigratinib or a pharmaceutically acceptable salt thereof. Also provided herein are methods of treating non-muscle invasive bladder cancer by administering to the patient infigratinib or a pharmaceutically acceptable salt thereof.

### CROSS-REFERENCE TO RELATED APPLICATIONS

This application claims the benefit of and priority to U.S. Patent Application No. 62/855,277, filed May 31, 2019, and U.S. Patent Application No. 62/906,194, filed Sep. 26, 2019, which are incorporated by reference herein in their entirety.

### BACKGROUND

In 2018, it was estimated that 150,350 new patients would be diagnosed with urinary system cancer: 81,190 urinary bladder; 65,340 kidney and renal pelvis; and, 3,820 ureter and other urinary organs. Excluding non-urothelial kidney cancers, approximately 5 to 10% of all urothelial carcinomas are upper tract urothelial carcinomas (UTUC). The incidence of UTUC is 2 to 3 times greater in men than women (Siegel et al., 2018; Roupret et al., 2015).

In contrast to invasive urinary bladder cancer (UCB), UTUC has a more aggressive clinical course. At the time of diagnosis, 60% of patients with UTUC have invasive cancer compared to 15% to 25% of patients with UCB (Roupret et al., 2015; Margulis et al., 2009). Thirty-six percent have regional disease and 9% distant disease (Raman et al., 2010). A large retrospective review of 1363 patients with UTUC who underwent radical nephroureterectomy (RNU) at 12 centers demonstrated that 28% of the total population had recurrence after RNU (Margulis et al., 2009).

To reduce the morbidity and mortality in patients with UTUC, neoadjuvant or adjuvant treatment is needed. The POUT study, a large randomized trial in UTUC supports the use of standard-of-care adjuvant cisplatin-based chemotherapy (Birtle et al., 2020). Because many patients with UTUC will have one remaining kidney following RNU and frequently have other significant co-morbid conditions, cisplatin-based therapy is not well tolerated (NCCN Guidelines Version 3, 2018). Renal function before and after RNU greatly limits the number of patients with UTUC who are eligible for platinum-based neoadjuvant or adjuvant therapy. Therefore, targeted therapies are needed for treating UTUC (Lane et al., 2010).

Despite demonstrated survival benefit for neoadjuvant treatment of invasive UCB, many patients with invasive UCB are unlikely to receive (neo)adjuvant cisplatin-based chemotherapy, due in part to cisplatin ineligibility (Porter et al., 2011). In addition, residual disease following neoadjuvant therapy is associated with a poor prognosis (Grossman et al., 2003). Therefore, there remains an unmet need for a substantial proportion of patients with invasive UCB who are ineligible or refuse to receive cisplatin-based adjuvant chemotherapy or who have residual disease following neoadjuvant therapy.

### SUMMARY

In one aspect, provided herein are methods of treating an upper tract urothelial carcinoma in a patient in need thereof, comprising administering to the patient an effective amount of infigratinib or a pharmaceutically acceptable salt thereof. In certain embodiments, the upper tract urothelial carcinoma is an invasive upper tract urothelial carcinoma. In certain embodiments, the upper tract urothelial carcinoma is a non-invasive upper tract urothelial carcinoma.

In certain embodiments, the patient is not eligible for treatment with a cisplatin-based chemotherapeutic therapy. In certain embodiments, the patient has previously been administered a cisplatin-based chemotherapy but has a residual carcinoma.

In certain embodiments, administering an effective amount of infigratinib, or a pharmaceutically acceptable salt thereof, occurs following a nephro-ureterectomy or a distal ureterectomy.

In certain embodiments, administering an effective amount of infigratinib, or a pharmaceutically acceptable salt thereof, to the patient has greater efficacy in treating the upper tract urothelial carcinoma compared to treating urothelial carcinoma of the bladder by administering the effective amount of infigratinib, or a pharmaceutically acceptable salt thereof, to a patient in need thereof.

In another aspect, provided herein are methods of treating a urothelial carcinoma in a patient in need thereof, comprising administering to the patient an effective amount of infigratinib, or a pharmaceutically acceptable salt thereof, wherein the patient has previously had a nephro-ureterectomy, a distal ureterectomy, or a cystectomy. In certain embodiments, the urothelial carcinoma is an invasive upper tract urothelial carcinoma or urothelial carcinoma of the bladder. In certain embodiments, the urothelial carcinoma is a non-invasive upper tract urothelial carcinoma or urothelial carcinoma of the bladder.

In certain embodiments, the patient is not eligible for treatment with a cisplatin-based chemotherapeutic therapy. In certain embodiments, the patient has previously been administered a cisplatin-based chemotherapy but has a residual carcinoma.

In certain embodiments, administering an effective amount of infigratinib, or a pharmaceutically acceptable salt thereof, comprises administering orally about 125 mg of infigratinib, or a pharmaceutically acceptable salt thereof, once daily.

In certain embodiments, administering an effective amount of infigratinib, or a pharmaceutically acceptable salt thereof, comprises a 28-day cycle, wherein about 125 mg of infigratinib, or a pharmaceutically acceptable salt thereof, is administered orally once daily to the patient for 3 consecutive weeks, and no infigratinib is administered for 1 week. In some embodiments, the about 125 mg of infigratinib, or a pharmaceutically acceptable salt thereof, is provided as a 100 mg unit dose and a 25 mg unit dose. In some embodiments, the about 125 mg of infigratinib, or a pharmaceutically acceptable salt thereof, is provided as a unit dose.

In certain embodiments, the urothelial carcinoma is histologically or cytologically confirmed.

In certain embodiments, the urothelial carcinoma has a FGFR3 mutation, gene rearrangement or fusion. In certain embodiments, the urothelial carcinoma has a FGFR3 mutation. In some embodiments, the FGFR3 mutation is selected from the group consisting of FGFR3 R248C, FGFR3 S249C, FGFR3 G372C, FGFR3 A393E, FGFR3 Y375C, FGFR3 K652M/T, FGFR3 K652E/Q, and combinations thereof.

In another aspect, provided herein are methods of treating non-muscle invasive bladder cancer in a patient in need thereof, comprising administering an effective amount of infigratinib, or a pharmaceutically acceptable salt thereof, wherein the patient has reoccurrence of the non-muscle invasive bladder cancer after previous administration of another therapy.

In certain embodiments, the previous administration of another therapy is a therapy for non-muscle invasive bladder cancer. In certain embodiments, the previous administration of another therapy is an administration of an immunotherapeutic agent. In some embodiments, the previous administration of an immunotherapeutic agent is a bacillus Calmette-Guerin-containing regimen.

In certain embodiments, the non-muscle invasive bladder cancer has a FGFR3 mutation, gene rearrangement or gene fusion. In certain embodiments, the non-muscle invasive bladder cancer has a FGFR3 mutation. In some embodiments, the FGFR3 mutation is selected from the group consisting of FGFR3 K650E, FGFR3 S249C, FGFR3 R248C, FGFR3 Y375C, FGFR3 G372C, FGFR3 S373C, FGFR3 A393E, FGFR3 A371A, FGFR3 I378C, FGFR3 L379L, FGFR3 G382R, and combinations thereof. In certain embodiments, the non-muscle invasive bladder cancer has a FGFR3 gene fusion. In some embodiments, the FGFR3 gene fusion comprises the FGFR3 gene fusion partner TACC3.

In certain embodiments, administering an effective amount of infigratinib, or a pharmaceutically acceptable salt thereof, comprises administering about 125 mg of infigratinib, or a pharmaceutically acceptable salt thereof, once daily.

In certain embodiments, administering an effective amount of infigratinib, or a pharmaceutically acceptable salt thereof comprises a 28-day cycle, wherein about 125 mg of infigratinib, or a pharmaceutically acceptable salt thereof, is administered orally once daily to the patient for 3 consecutive weeks, and no infigratinib is administered for 1 week.

In certain embodiments, the about 125 mg of infigratinib or a pharmaceutically acceptable salt thereof is provided as a 100 mg unit dose and a 25 mg unit dose. In certain embodiments, the about 125 mg of infigratinib or a pharmaceutically acceptable salt thereof is provided as a unit dose.

In certain embodiments, the about 125 mg of infigratinib or a pharmaceutically acceptable salt thereof is administered orally to the patient.

In certain embodiments, the effective amount of infigratinib or a pharmaceutically acceptable salt thereof is administered to the patient via local administration. In some embodiments, the effective amount of infigratinib or a pharmaceutically acceptable salt thereof is administered to the patient intratumorally. In some embodiments, the effective amount of infigratinib or a pharmaceutically acceptable salt thereof is administered to the patient intravesically. In certain embodiments, the effective amount of infigratinib or a pharmaceutically acceptable salt thereof is delivered via insertion of a controlled release, implantable device into the patient's bladder. In certain embodiments, the effective amount of infigratinib or a pharmaceutically acceptable salt thereof is delivered via insertion of a controlled release, implantable device into the patient's ureter. In certain embodiments, the effective amount of infigratinib or a pharmaceutically acceptable salt thereof is delivered via insertion of a controlled release, implantable device into the patient's renal pelvis.

In certain embodiments, the controlled release, implantable device is a dual-lumen silicon tube comprising a superelastic wireform. In certain embodiments, the controlled release, implantable device is a gel.

In certain embodiments, the method further comprising administering an effective amount of a second therapeutic agent to the patient. In some embodiments, the effective amount of the second therapeutic agent is administered to the patient via local administration. In certain embodiments, the effective amount of the second therapeutic agent is administered to the patient intravesically. In some embodiments, the second therapeutic agent is gemcitabine or a pharmaceutically acceptable salt thereof.

In another aspect, provided herein are methods of treating an upper tract urothelial carcinoma in a patient in need thereof, comprising administering to the patient an effective amount of infigratinib or a pharmaceutically acceptable salt thereof, wherein the effective amount of infigratinib or a pharmaceutically acceptable salt thereof is administered as a neoadjuvant therapy.

In certain embodiments, the upper tract urothelial carcinoma has a FGFR3 mutation, gene rearrangement or gene fusion.

In another aspect, provided herein are methods of treating a patient in need thereof having an upper tract urothelial carcinoma with at least one FGFR3 mutation, gene rearrangement or gene fusion, the method comprising: (i) obtaining a sample from the patient; (ii) analyzing the sample for the presence of the at least one FGFR3 mutation, gene rearrangement or gene fusion; and (iii) administering to the patient an effective amount of infigratinib or a pharmaceutically acceptable salt thereof, wherein the effective amount of infigratinib or a pharmaceutically acceptable salt thereof is administered as a neoadjuvant therapy.

In certain embodiments, the urothelial carcinoma has a FGFR3 mutation. In some embodiments, the FGFR3 mutation is selected from the group consisting of FGFR3 R248C, FGFR3 S249C, FGFR3 G372C, FGFR3 A393E, FGFR3 Y375C, FGFR3 K652M/T, FGFR3 K652E/Q, and combinations thereof.

In certain embodiments, the upper tract urothelial carcinoma is a low-grade upper tract urothelial carcinoma. In certain embodiments, the upper tract urothelial carcinoma is a high-grade upper tract urothelial carcinoma.

In certain embodiments, the patient is not eligible for treatment with a cisplatin-based neoadjuvant chemotherapeutic therapy.

In certain embodiments, administering an effective amount of infigratinib, or a pharmaceutically acceptable salt thereof, comprises administering orally about 125 mg of infigratinib, or a pharmaceutically acceptable salt thereof, once daily.

In certain embodiments, administering an effective amount of infigratinib, or a pharmaceutically acceptable salt thereof comprises a 28-day cycle, wherein about 125 mg of infigratinib, or a pharmaceutically acceptable salt thereof, is administered orally once daily to the patient for 3 consecutive weeks, and no infigratinib is administered for 1 week. In certain embodiments, the effective amount of infigratinib, or a pharmaceutically acceptable salt thereof is administered to the patient for two consecutive 28-days cycles.

In certain embodiments, the about 125 mg of infigratinib or a pharmaceutically acceptable salt thereof is provided as a 100 mg unit dose and a 25 mg unit dose. In certain embodiments, the about 125 mg of infigratinib or a pharmaceutically acceptable salt thereof is provided as a unit dose.

In certain embodiments, the method further comprises the patient undergoing a nephro-ureterectomy or a ureterectomy within 8 weeks of commencing the neoadjuvant therapy.

In another aspect, provided herein are methods of identifying a patient for treatment of an upper tract urothelial carcinoma with an effective amount of infigratinib or a pharmaceutically acceptable salt thereof, comprising: testing a sample obtained from the patient after administration of the effective amount of infigratinib or a pharmaceutically acceptable salt thereof to measure gene expression of at least one FGFR3 biomarker, wherein detection of an alteration in the level of expression of the at least one FGFR3 biomarker compared to a baseline gene expression measurement is indicative of the candidacy of the patient for treatment, and wherein the baseline gene expression measurement is the gene expression measured in the patient prior to administration of the effective amount of infigratinib or a pharmaceutically acceptable salt thereof.

In another aspect, provided herein are methods for monitoring the response of a patient to treatment by an effective amount of infigratinib or a pharmaceutically acceptable salt thereof for an upper tract urothelial carcinoma, comprising: testing a sample obtained from the patient after administration of the effective amount of infigratinib or a pharmaceutically acceptable salt thereof to measure gene expression of at least one FGFR3 biomarker, wherein detection of an alteration in the level of expression of the at least one FGFR3 biomarker compared to a baseline gene expression measurement is indicative of the response of the patient to the treatment, and wherein the baseline gene expression measurement is the gene expression measured in the patient prior to administration of the effective amount of infigratinib or a pharmaceutically acceptable salt thereof.

In another aspect, provided herein are methods of identifying a patient for treatment of an upper tract urothelial carcinoma with an effective amount of infigratinib or a pharmaceutically acceptable salt thereof, comprising: testing a sample obtained from the patient after administration of the effective amount of infigratinib or a pharmaceutically acceptable salt thereof to measure the allele frequency of at least one FGFR3 biomarker in the patients cell-free DNA (cfDNA), wherein detection of the at least one FGFR3 biomarker at a lower variant allele frequency in the patient's cfDNA compared to a baseline allele frequency of the at least one FGFR3 biomarker is indicative of the candidacy of the patient for treatment, and wherein the baseline allele frequency measurement is the allele frequency measured in the patient's cfDNA prior to administration of the effective amount of infigratinib or a pharmaceutically acceptable salt thereof.

In another aspect, provided herein are methods for monitoring the response of a patient to treatment by an effective amount of infigratinib or a pharmaceutically acceptable salt thereof for an upper tract urothelial carcinoma, comprising: testing a sample obtained from the patient after administration of the effective amount of infigratinib or a pharmaceutically acceptable salt thereof to measure the allele frequency of at least one FGFR3 biomarker in the patient's cell-free DNA (cfDNA), wherein detection of the at least one FGFR3 biomarker at a lower variant allele frequency in the patient's cfDNA compared to a baseline allele frequency of the at least one FGFR3 biomarker is indicative of the response of the patient to the treatment, and wherein the baseline allele frequency measurement is the allele frequency measured in the patient's cfDNA prior to administration of the effective amount of infigratinib or a pharmaceutically acceptable salt thereof.

### BRIEF DESCRIPTION OF THE DRAWINGS

FIG. 1A depicts the frequency of mutations in FGFR3 observed in tumor tissue of upper tract urothelial carcinoma patients.

FIG. 1B depicts the frequency of mutations in FGFR3 observed in tumor tissue of urothelial carcinoma of the bladder patients.

FIG. 2 is an overlay of the progression-free survival rates of upper tract urothelial carcinoma patients and urothelial carcinoma of the bladder patients treated with infigratinib.

FIG. 3 is an overlay of the overall survival rates of upper tract urothelial carcinoma patients and urothelial carcinoma of the bladder patients treated with infigratinib.

FIG. 4 depicts a comparison of the frequency of variants in the cfDNA of upper tract urothelial carcinoma patients and urothelial carcinoma of the bladder patients.

FIG. 5 depicts the median variant allele frequency for FGFR3 genomic alterations in tumor tissue and cfDNA for upper tract urothelial carcinoma patients and urothelial carcinoma of the bladder patients.

FIG. 6 depicts oncoplots of cfDNA genomic profiles in upper tract urothelial carcinoma patients and urothelial carcinoma of the bladder patients.

### DETAILED DESCRIPTION

As generally described herein, the present disclosure provides methods of treating an upper tract urothelial carcinoma in a patient in need thereof, for example, when the patient has an invasive upper tract urothelial carcinoma. The present disclosure also provides methods of treating a urothelial carcinoma (e.g., an invasive upper tract urothelial carcinoma or urothelial carcinoma of the bladder) in a patient in need thereof, for example, when the patient has previously had a nephro-ureterectomy, a distal ureterectomy, or a cystectomy.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, “pharmaceutical composition” or “pharmaceutical formulation” refers to the combination of an active agent with a carrier, inert or active, making the composition especially suitable for diagnostic or therapeutic use in vivo or ex vivo.

“Pharmaceutically acceptable” means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present invention (e.g., infigratinib), which salt is compatible with pharmaceutical administration.

As is known to those of skill in the art, “salts” of compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW₄⁺, wherein W is C₁₋₄ alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, K⁺, Ca²⁺, NH₄⁺, and NW₄⁺ (where W can be a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients, see Martin, Remington's Pharmaceutical Sciences, 15th Ed., Mack Publ. Co., Easton, Pa. (1975).

The term “AUC” refers to the area under the time/plasma concentration curve after administration of the pharmaceutical composition. AUC_{0-infinity} denotes the area under the plasma concentration versus time curve from time 0 to infinity; AUC₀₋ₜ denotes the area under the plasma concentration versus time curve from time 0 to time t. It should be appreciated that AUC values can be determined by known methods in the art.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

The term “Cₘₐₓ” refers to the maximum concentration of a therapeutic agent (e.g., infigratinib) in the blood (e.g., plasma) following administration of the pharmaceutical composition.

The term “tₘₐₓ” refers to the time in hours when Cₘₐₓ is achieved following administration of the pharmaceutical composition comprising a therapeutic agent (e.g., infigratinib).

As used herein, “solid dosage form” means a pharmaceutical dose(s) in solid form, e.g., tablets, capsules, granules, powders, sachets, reconstitutable powders, dry powder inhalers and chewables.

As used herein, “administering” means oral administration, administration as a suppository, topical contact, intravenous administration, parenteral administration, intraperitoneal administration, intramuscular administration, intralesional administration, intrathecal administration, intracranial administration, intranasal administration or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent, chemotherapeutic, or treatment for a neurodegenerative disease). Infigratinib, or a pharmaceutically acceptable salt thereof, can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (e.g., “therapeutic treatment”).

In general, an “effective amount” of a compound refers to an amount sufficient to elicit the desired biological response, e.g., to treat upper tract urothelial carcinoma or non-muscle invasive bladder cancer. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject.

### Infigratinib

Infigratinib, as depicted in formula (I), is a selective and ATP-competitive pan-fibroblast growth factor receptor (FGFR) kinase inhibitor, also known as 3-(2,6-dichloro-3,5-dimethoxyphenyl)-1-{6-[4-(4-ethyl-1-piperazin-1-yl)phenylamino]-pyrimidinyl-4-yl}-1-methylurea. Infigratinib selectively inhibits the kinase activity of FGFR1, FGFR2, FGFR3, and FGFR4.

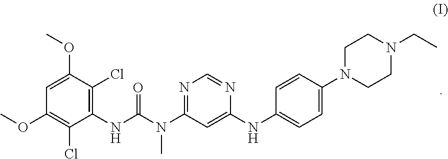 [SMILES: CCN1CCN(c2ccc(Nc3cc(N(C)C(=O)Nc4c(Cl)c(OC)cc(OC)c4Cl)ncn3)cc2)CC1]

A method of chemically synthesizing infigratinib (including Example 1 provided herein), several crystalline and amorphous forms of infigratinib (including the anhydrous crystalline monophosphate salt described herein) and methods of preparing said forms (including Example 2 provided herein) were described in U.S. Pat. No. 9,067,896, which is incorporated by reference in its entirety herein.

In one aspect, provided herein is a method of administering infigratinib, or a pharmaceutically acceptable salt thereof, for the treatment of an upper tract urothelial carcinoma in a patient in need thereof. In certain embodiments, the patient has previously had a nephro-ureterectomy, a distal ureterectomy, or a cystectomy. In certain embodiments, the infigratinib, or a pharmaceutically acceptable salt thereof, is administered prior to the patient undergoing a nephro-ureterectomy, a distal ureterectomy, or a cystectomy.

In another aspect, provided herein is a method of administering infigratinib, or a pharmaceutically acceptable salt thereof, for the treatment of a urothelial carcinoma (e.g., an invasive upper tract urothelial carcinoma or urothelial carcinoma of the bladder) in a patient in need thereof, wherein the patient has previously had a nephro-ureterectomy, a distal ureterectomy, or a cystectomy.

In another aspect, provided herein is a method of administering infigratinib, or a pharmaceutically acceptable salt thereof, for the treatment of non-muscle invasive bladder cancer.

In another aspect, provided herein is a method of administering infigratinib, or a pharmaceutically acceptable salt thereof, for the treatment of non-muscle invasive bladder cancer, wherein the patient has reoccurrence of the non-muscle invasive bladder cancer after previous administration of another therapy.

In certain embodiments, provided herein is a method of administering a pharmaceutically acceptable salt of infigratinib for the treatment of an upper tract urothelial carcinoma in a patient in need thereof. In certain embodiments, provided herein is a method of administering a pharmaceutically acceptable salt of infigratinib for the treatment of a urothelial carcinoma (e.g., an invasive upper tract urothelial carcinoma or urothelial carcinoma of the bladder) in a patient in need thereof, wherein the patient has previously had a nephro-ureterectomy, a distal ureterectomy, or a cystectomy.

In certain embodiments, provided herein is a method of administering a pharmaceutically acceptable salt of infigratinib for the treatment of non-muscle invasive bladder cancer. In certain embodiments, provided herein is a method of administering a pharmaceutically acceptable salt of infigratinib for the treatment of non-muscle invasive bladder cancer, wherein the patient has reoccurrence of the non-muscle invasive bladder cancer after previous administration of another therapy.

In certain embodiments, the pharmaceutically acceptable salt of infigratinib is a monophosphate salt. The monophosphate salt of infigratinib may also be referred to as BGJ398.

In some embodiments, the monophosphate salt of infigratinib is an anhydrous crystalline monophosphate salt. In some embodiments, the anhydrous crystalline monophosphate salt has an X-ray powder diffraction (XRPD) pattern comprising a characteristic peak, in terms of 2θ, at about 15.0° or 15.0°±0.2°. In some embodiments, the X-ray powder diffraction pattern of the anhydrous crystalline monophosphate salt further comprises one or more characteristic peaks, in terms of 2θ, selected from peaks at about 13.7°±0.2°, about 16.8°±0.2°, about 21.3°±0.2° and about 22.4°±0.2°. In some embodiments, the X-ray powder diffraction pattern of the anhydrous crystalline monophosphate salt further comprises one or more characteristic peaks, in terms of 2θ, selected from peaks at about 9.2°, about 9.6°, about 18.7°, about 20.0°, about 22.9°, and about 27.2°. In some embodiment, the anhydrous crystalline monophosphate salt has an XRPD pattern comprising at least three characteristic peaks, in terms of 2θ, selected from the peaks at about 13.7°, about 15°, about 16.8, about 21.3° and about 22.4°. In some embodiments, the X-ray powder diffraction pattern for the anhydrous crystalline monophosphate salt may comprise one, two, three, four, five, six, seven, eight, nine, ten or eleven characteristic peaks, in terms of 2θ, selected from the peaks at about 9.2°, about 9.6°, about 13.7°, about 15°, about 16.8°, about 18.7°, about 20.0°, about 21.3° and about 22.4°, about 22.9°, and about 27.2.

### Pharmaceutical Compositions

Pharmaceutical compositions of infigratinib and methods of preparing said pharmaceutical compositions (e.g., including Example 3 described herein) were described in U.S. Patent Application Publication No. 2017/0007602, which is incorporated by reference in its entirety herein.

In one aspect, provided herein is a method of administering a pharmaceutical composition comprising infigratinib, or a pharmaceutically acceptable salt thereof, and a pharmaceutically acceptable excipient, for the treatment of an upper tract urothelial carcinoma in a patient in need thereof. In certain embodiments, the patient has previously had a nephro-ureterectomy, a distal ureterectomy, or a cystectomy. In certain embodiments, the infigratinib, or a pharmaceutically acceptable salt thereof, is administered prior to the patient undergoing a nephro-ureterectomy, a distal ureterectomy, or a cystectomy.

In another aspect, provided herein is a method of administering a pharmaceutical composition comprising infigratinib, or a pharmaceutically acceptable salt thereof, and a pharmaceutically acceptable excipient, for the treatment of a urothelial carcinoma (e.g., an invasive upper tract urothelial carcinoma or urothelial carcinoma of the bladder) in a patient in need thereof, wherein the patient has previously had a nephro-ureterectomy, a distal ureterectomy, or a cystectomy.

In another aspect, provided herein is a method of administering a pharmaceutical composition comprising infigratinib, or a pharmaceutically acceptable salt thereof, and a pharmaceutically acceptable excipient, for the treatment of non-muscle invasive bladder cancer in a patient in need thereof.

In another aspect, provided herein is a method of administering a pharmaceutical composition comprising infigratinib, or a pharmaceutically acceptable salt thereof, and a pharmaceutically acceptable excipient, for the treatment of non-muscle invasive bladder cancer in a patient in need thereof, wherein the patient has reoccurrence of the non-muscle invasive bladder cancer after previous administration of another therapy.

In certain embodiments, a pharmaceutical composition of the present teachings comprises:
(a) from about 20% to about 60% infigratinib, or a pharmaceutically acceptable salt thereof, by weight in its free base form;
(b) from about 0.5% to about 5% by weight of hydroxypropylmethylcellulose;
(c) from about 1% to about 4% by weight of crosslinked polyvinylpyrrolidone; and
(d) a filler selected from the group consisting of a cellulose, lactose, mannitol, and combinations thereof;
wherein the weight percentages are based on the total weight of the pharmaceutical composition.

In certain embodiments, the pharmaceutical composition comprises from about 30%-45% of infigratinib, or a pharmaceutically acceptable salt thereof, by weight in its free base form.

In certain embodiments, the pharmaceutical composition comprises from about 2% to about 4% of hydroxypropylmethylcellulose. In certain embodiments, the pharmaceutical composition comprises from about 2% to about 4% of crosslinked polyvinylpyrrolidone.

In certain embodiments, the pharmaceutical composition further comprises one or more of:
(e) from about 10% to about 95% of one or more fillers, by weight based on the total weight of the pharmaceutical composition;
(f) from about 0.1% to about 3% of one or more lubricants, by weight based on the total weight of the pharmaceutical composition; and
(g) from about 0.1% to about 2% of one or more glidants, by weight based on the total weight of the pharmaceutical composition.

In certain embodiments, the one or more fillers is selected from the group consisting of microcrystalline cellulose, lactose, and/or mannitol.

In certain embodiments, the one or more lubricants in the pharmaceutical composition is present in an amount of from about 0.2% to about 2%, by weight based on the total weight of the pharmaceutical composition. In some embodiments, the one or more lubricants is magnesium stearate.

In certain embodiments, the one or more glidants is present in the pharmaceutical formulation in an amount of from about 0.1% to about 0.5%, by weight based on the total weight of the pharmaceutical composition. In some embodiments, the one or more glidants is colloidal silicon dioxide (colloidal silica).

In certain embodiments, the amount of infigratinib, or a pharmaceutically acceptable salt thereof, in the pharmaceutical composition is from about 25 mg to about 150 mg, about 50 mg to about 150 mg, about 75 mg to about 150 mg, about 100 mg to about 150 mg, about 125 mg to about 150 mg, about 25 mg to about 125 mg, about 25 mg to about 100 mg, about 25 mg to about 75 mg, about 25 mg to about 50 mg, about 50 mg to about 125 mg, about 50 mg to about 100 mg, about 50 mg to about 75 mg, about 75 mg to about 125 mg, about 75 mg to about 100 mg, or about 100 mg to about 125 mg. In some embodiments, the amount of infigratinib, or a pharmaceutically acceptable salt thereof, in the pharmaceutical composition is from about 100 mg to about 150 mg of infigratinib or a pharmaceutically acceptable salt thereof.

In certain embodiments, the amount of infigratinib, or a pharmaceutically acceptable salt thereof, in the pharmaceutical composition is about 25 mg, about 50 mg, about 75 mg, about 100 mg, about 125 mg, about 150 mg, about 175 mg, or about 200 mg. In some embodiments, the amount of infigratinib, or a pharmaceutically acceptable salt thereof, in the pharmaceutical composition is about 125 mg. In some embodiments, the amount of infigratinib, or a pharmaceutically acceptable salt thereof, in the pharmaceutical composition is about 100 mg. In some embodiments, the amount of infigratinib, or a pharmaceutically acceptable salt thereof, in the pharmaceutical composition is about 25 mg.

In another aspect, provided herein is a pharmaceutical composition comprising about 125 mg infigratinib, or a pharmaceutically acceptable salt thereof; and a pharmaceutically acceptable excipient, for the treatment of an upper tract urothelial carcinoma in a patient in need thereof.

In another aspect, provided herein is a pharmaceutical composition comprising about 125 mg infigratinib, or a pharmaceutically acceptable salt thereof; and a pharmaceutically acceptable excipient, for the treatment of a urothelial carcinoma in a patient in need thereof, wherein the patient has previously had a nephro-ureterectomy, a distal ureterectomy, or a cystectomy.

In another aspect, provided herein is a pharmaceutical composition comprising about 125 mg infigratinib, or a pharmaceutically acceptable salt thereof; and a pharmaceutically acceptable excipient, for the treatment of non-muscle invasive bladder cancer in a patient in need thereof.

In another aspect, provided herein is a pharmaceutical composition comprising about 125 mg infigratinib, or a pharmaceutically acceptable salt thereof; and a pharmaceutically acceptable excipient, for the treatment of non-muscle invasive bladder cancer in a patient in need thereof, wherein the patient has reoccurrence of the non-muscle invasive bladder cancer after previous administration of another therapy.

In certain embodiments, the pharmaceutical compositions comprise an effective amount of a pharmaceutically acceptable salt of infigratinib. In some embodiments, the pharmaceutically acceptable salt of infigratinib is a monophosphate salt. In some embodiments, the pharmaceutically acceptable salt of infigratinib is an anhydrous monophosphate salt. In some embodiments, the pharmaceutically acceptable salt of infigratinib is an anhydrous monophosphate salt in a polymorphic form characterized by an X-ray powder diffraction (XRPD) peak (2 Theta) at 15.0°±0.2° (and can include the other XRPD peaks of this form as described herein).

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In some embodiments, the pharmaceutical compositions disclosed herein are administered orally.

The pharmaceutical compositions provided herein may also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc., or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions provided herein may be presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions.

In certain embodiments, the pharmaceutical compositions provided herein are administered to the patient as a solid dosage form. In some embodiments, the solid dosage form is a capsule.

In certain embodiments, the compounds provided herein can be administered as the sole active agent, or they can be administered in combination with other active agents.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### Methods of Use and Treatment

### Treatment of Upper Tract Urothelial Carcinoma

Urothelial carcinomas, which may also be referred to as transitional cell carcinomas, are a class of cancers that typically occur in the urinary system. These cancers may occur, for example, in the bladder, renal pelvis, ureters and/or urethra.

In one aspect, provided herein are methods of treating an upper tract urothelial carcinoma in a patient in need thereof.

In certain embodiments, provided herein are methods of treating upper tract urothelial carcinoma in a patient in need thereof, comprising administering to the patient an effective amount of infigratinib, or a pharmaceutically acceptable salt thereof.

In certain embodiments, the upper tract urothelial carcinoma is an invasive upper tract urothelial carcinoma. In some embodiments, the invasive upper tract urothelial carcinoma is located in the renal calcyces, renal pelvis and/or ureters. In certain embodiments, the upper tract urothelial carcinoma is a non-invasive upper tract urothelial carcinoma.

In certain embodiments, the patient is not eligible for treatment with a cisplatin-based chemotherapeutic therapy. In certain embodiments, the patient has previously had cisplatin-based chemotherapy but has a residual carcinoma.

In certain embodiments, administering an effective amount of infigratinib, or a pharmaceutically acceptable salt thereof, occurs prior to the patient undergoing a nephro-ureterectomy or a distal ureterectomy. In certain embodiments, administering an effective amount of infigratinib, or a pharmaceutically acceptable salt thereof, occurs following a nephro-ureterectomy or a distal ureterectomy. In certain embodiments, administering an effective amount of infigratinib, or a pharmaceutically acceptable salt thereof, occurs within 120 days following a nephro-ureterectomy or a distal ureterectomy.

In certain embodiments, administering an effective amount of infigratinib, or a pharmaceutically acceptable salt thereof, to the patient has greater efficacy in treating the upper tract urothelial carcinoma compared to treating urothelial carcinoma of the bladder by administering the effective amount of infigratinib, or a pharmaceutically acceptable salt thereof, to a patient in need thereof.

In certain embodiments, the upper tract urothelial carcinoma is histologically or cytologically confirmed.

In certain embodiments, the upper tract urothelial carcinoma has a FGFR1 mutation, gene rearrangement or gene fusion. In certain embodiments, the upper tract urothelial carcinoma has a FGFR1 gene fusion. In some embodiments, the FGFR1 gene fusion comprises a FGFR1 gene fusion partner selected from the group consisting of BAG4, ERLIN2, NTM, FGFR10P2, TACC3, and TRP.

In certain embodiments, the upper tract urothelial carcinoma has a FGFR2 mutation, gene rearrangement or gene fusion. In certain embodiments, the upper tract urothelial carcinoma has a FGFR2 gene fusion. In some embodiments, the FGFR2 gene fusion comprises a FGFR2 gene fusion partner selected from the group consisting of 10Q26.13, AFF1, AFF3, AFF4, AHCYL1, ALDH1L2, ARFIP1, BAG4, BAIAP2L1, BICC1, C10orf118, C10orf68, C7, CASC15, CASP7, CCDC147, CCDC6, CELF2, CIT, COL14A1, CREB5, CREM, DNAJC12, ERLIN2, HOOK1, INA, KCTD1, KIAA1217, KIAA1598, KIAA1967, KIFC3, MGEA5, NCALD, NOL4, NPM1, NRAP, OFD1, OPTN, PARK2, PAWR, PCMI, PDHX, PHLDB2, PPAPDC1A, PPHLN1, RASAL2, SFMBT2, SLC45A3, SLMAP, SLMAP2, SORBS1, STK26, STK3, TACC1, TACC2, TACC3, TBC1D1, TEL, TFEC, TRA2B, UBQLN1, VCL, WAC, ZMYM4, and FGFROP2.

In certain embodiments, the upper tract urothelial carcinoma has a FGFR3 mutation, gene rearrangement or gene fusion. In certain embodiments, the upper tract urothelial carcinoma has a FGFR3 gene fusion. In some embodiments, the FGFR3 gene fusion comprises a FGFR3 gene fusion partner selected from the group consisting of BAIAP2L1, JAKMIP1, TACC3, TNIP2, and WHSC1.

In certain embodiments, the patient undergoes molecular prescreening, for example using next generation sequencing, circulating tumor DNA analysis or a fluorescence in situ hybridization assay, to determine whether the upper tract urothelial carcinoma has a FGFR1, FGFR2, or FGFR3 mutation, gene rearrangement or gene fusion. In some embodiments, the molecular prescreening occurs prior to administration of the effective amount of infigratinib, or a pharmaceutically acceptable salt thereof. In some embodiments, the molecular prescreening to occurs prior to the previous administration of a cisplatin-based chemotherapeutic therapy.

In certain embodiments, the upper tract urothelial carcinoma has a FGFR1, FGFR2 and/or FGFR3 mutation. In certain embodiments, the upper tract urothelial carcinoma has a FGFR1 mutation. In certain embodiments, the upper tract urothelial carcinoma has a FGFR2 mutation. In certain embodiments, the upper tract urothelial carcinoma has a FGFR3 mutation.

In certain embodiments, the FGFR1 mutation is selected from the group consisting of FGFR1 G818R, FGFR1 K656E, FGFR1 N546K, FGFR1 R445W, FGFR1 T141R, and combinations thereof.

In certain embodiments, the FGFR2 mutation is selected from the group consisting of FGFR2 D471N, FGFR2 A315T, FGFR2 D336N, FGFR2 P253R, FGFR2 S252W, FGFR2 Y375C, FGFR2 I547V, FGFR2 K659E, FGFR2 N549K, FGFR2 N549S, FGFR2 N549Y, FGFR2 V395D, FGFR2 C382R, FGFR2 E565A, FGFR2 K641R, FGFR2 K659M, FGFR2 L617V, FGFR2 N549H, FGFR2 N550K, FGFR2 V564F, and combinations thereof.

In certain embodiments, the FGFR3 mutation is selected from the group consisting of FGFR3 A391E, FGFR3 A393E, FGFR3 D785Y, FGFR3 E627K, FGFR3 G370C, FGFR3 G372C, FGFR3 G380R, FGFR3 K650E, FGFR3 K652E/Q, FGFR3 K650M, FGFR3 K652M/T, FGFR3 K650N, FGFR3 K650T, FGFR3 K652E, FGFR3 N540S, FGFR3 R248C, FGFR3 R399C, FGFR3 S131L, FGFR3 S249C, FGFR3 S249C, FGFR3 S371C, FGFR3 V555M, FGFR3 V6771, FGFR3 Y373C, FGFR3 Y375C, and combinations thereof. In some embodiments, the FGFR3 mutation is selected from the group consisting of FGFR3 R248C, FGFR3 S249C, FGFR3 G372C, FGFR3 A393E, FGFR3 Y375C, FGFR3 K652M/T, FGFR3 K652E/Q, and combinations thereof.

In another aspect, provided herein are methods for treating an upper tract urothelial carcinoma in a patient in need thereof, comprising administering to the patient an effective amount of infigratinib or a pharmaceutically acceptable salt thereof, wherein the effective amount of infigratinib or a pharmaceutically acceptable salt thereof is administered as a neoadjuvant therapy.

In certain embodiments, the upper tract urothelial carcinoma has a FGFR3 mutation, gene rearrangement or gene fusion.

In another aspect, provided herein are methods for treating a patient in need thereof having an upper tract urothelial carcinoma with at least one FGFR3 mutation, gene rearrangement or gene fusion, the method comprising:
(i) obtaining a sample from the patient;
(ii) analyzing the sample for the presence of the at least one FGFR3 mutation, gene rearrangement or gene fusion; and
(iii) administering to the patient an effective amount of infigratinib or a pharmaceutically acceptable salt thereof,
   wherein the effective amount of infigratinib or a pharmaceutically acceptable salt thereof is administered as a neoadjuvant therapy.

In certain embodiments, the sample obtained from the patient is a sample obtained from the patient using a method selected from the group consisting of a selective upper tract washing, fine needle aspirates, core needle biopsy, brush biopsy, urine cell free DNA, blood cell free DNA, and other cytology samples (for cytology sampling of metastatic sites such as pleural effusions, etc.).

In another aspect, provided herein are methods for treating a patient in need thereof having an upper tract urothelial carcinoma with at least one FGFR3 mutation, gene rearrangement or gene fusion, the method comprising:
(i) obtaining a sample from the patient, wherein the sample is obtained from the patient using a selective upper tract washing;
(ii) analyzing the sample for the presence of the at least one FGFR3 mutation, gene rearrangement or gene fusion; and
(iii) administering to the patient an effective amount of infigratinib or a pharmaceutically acceptable salt thereof,
   wherein the effective amount of infigratinib or a pharmaceutically acceptable salt thereof is administered as a neoadjuvant therapy.

In certain embodiments, the upper tract urothelial carcinoma is a low-grade upper tract urothelial carcinoma. In certain embodiments, the upper tract urothelial carcinoma is a high-grade upper tract urothelial carcinoma.

In certain embodiments, the patient is not eligible for treatment with a cisplatin-based neoadjuvant chemotherapeutic therapy.

In certain embodiments, the method further comprises the patient undergoing a nephro-ureterectomy or a ureterectomy within 5 weeks, 6 weeks, 7 weeks, 8 weeks, 9 weeks, or 10 weeks of commencing the neoadjuvant therapy. In some embodiments, the method further comprises the patient undergoing a nephro-ureterectomy or a ureterectomy within 8 weeks of commencing the neoadjuvant therapy.

In another aspect, provided herein are methods of identifying a patient for treatment of an upper tract urothelial carcinoma with an effective amount of infigratinib or a pharmaceutically acceptable salt thereof, comprising:

testing a sample obtained from the patient after administration of the effective amount of infigratinib or a pharmaceutically acceptable salt thereof to measure gene expression of at least one FGFR3 biomarker, wherein detection of an alteration in the level of expression of the at least one FGFR3 biomarker compared to a baseline gene expression measurement is indicative of the candidacy of the patient for treatment, and wherein the baseline gene expression measurement is the gene expression measured in the patient prior to administration of the effective amount of infigratinib or a pharmaceutically acceptable salt thereof.

In another aspect, provided herein are methods for monitoring the response of a patient to treatment by an effective amount of infigratinib or a pharmaceutically acceptable salt thereof for an upper tract urothelial carcinoma, comprising:

testing a sample obtained from the patient after administration of the effective amount of infigratinib or a pharmaceutically acceptable salt thereof to measure gene expression of at least one FGFR3 biomarker,
wherein detection of an alteration in the level of expression of the at least one FGFR3 biomarker compared to a baseline gene expression measurement is indicative of the response of the patient to the treatment, and
wherein the baseline gene expression measurement is the gene expression measured in the patient prior to administration of the effective amount of infigratinib or a pharmaceutically acceptable salt thereof.

In certain embodiments, the sample obtained from the patient is a voided urine sample. In certain embodiments, the sample obtained from the patient is a blood sample. In certain embodiments, the sample obtained from the patient is a sample obtained from the patient using a selective upper tract washing.

In another aspect, provided herein are methods of identifying a patient for treatment of an upper tract urothelial carcinoma with an effective amount of infigratinib or a pharmaceutically acceptable salt thereof, comprising:

testing a sample obtained from the patient after administration of the effective amount of infigratinib or a pharmaceutically acceptable salt thereof to measure gene expression of at least one FGFR3 biomarker,
wherein detection of an alteration in the level of expression of the at least one FGFR3 biomarker compared to a baseline gene expression measurement is indicative of the candidacy of the patient for treatment, and
wherein the baseline gene expression measurement is the gene expression measured in the patient prior to administration of the effective amount of infigratinib or a pharmaceutically acceptable salt thereof.

In certain embodiments, the sample obtained from the patient is a sample obtained from the patient using a method selected from the group consisting of a selective upper tract washing, fine needle aspirates, core needle biopsy, brush biopsy, urine cell free DNA, blood cell free DNA, and other cytology samples (for cytology sampling of metastatic sites such as pleural effusions, etc.).

In another aspect, provided herein are methods of identifying a patient for treatment of an upper tract urothelial carcinoma with an effective amount of infigratinib or a pharmaceutically acceptable salt thereof, comprising:

testing a sample obtained from the patient after administration of the effective amount of infigratinib or a pharmaceutically acceptable salt thereof to measure gene expression of at least one FGFR3 biomarker, wherein the sample is obtained from the patient using a selective upper tract washing,
wherein detection of an alteration in the level of expression of the at least one FGFR3 biomarker compared to a baseline gene expression measurement is indicative of the candidacy of the patient for treatment, and
wherein the baseline gene expression measurement is the gene expression measured in the patient prior to administration of the effective amount of infigratinib or a pharmaceutically acceptable salt thereof.

In another aspect, provided herein are methods for monitoring the response of a patient to treatment by an effective amount of infigratinib or a pharmaceutically acceptable salt thereof for an upper tract urothelial carcinoma, comprising:

testing a sample obtained from the patient after administration of the effective amount of infigratinib or a pharmaceutically acceptable salt thereof to measure gene expression of at least one FGFR3 biomarker,
wherein detection of an alteration in the level of expression of the at least one FGFR3 biomarker compared to a baseline gene expression measurement is indicative of the response of the patient to the treatment, and
wherein the baseline gene expression measurement is the gene expression measured in the patient prior to administration of the effective amount of infigratinib or a pharmaceutically acceptable salt thereof.

In certain embodiments, the sample obtained from the patient is a sample obtained from the patient using a method selected from the group consisting of a selective upper tract washing, fine needle aspirates, core needle biopsy, brush biopsy, urine cell free DNA, blood cell free DNA, and other cytology samples (for cytology sampling of metastatic sites such as pleural effusions, etc.).

In another aspect, provided herein are methods for monitoring the response of a patient to treatment by an effective amount of infigratinib or a pharmaceutically acceptable salt thereof for an upper tract urothelial carcinoma, comprising:

testing a sample obtained from the patient after administration of the effective amount of infigratinib or a pharmaceutically acceptable salt thereof to measure gene expression of at least one FGFR3 biomarker, wherein the sample is obtained from the patient using a selective upper tract washing,
wherein detection of an alteration in the level of expression of the at least one FGFR3 biomarker compared to a baseline gene expression measurement is indicative of the response of the patient to the treatment, and
wherein the baseline gene expression measurement is the gene expression measured in the patient prior to administration of the effective amount of infigratinib or a pharmaceutically acceptable salt thereof.

In another aspect, provided herein are methods of identifying a patient for treatment of an upper tract urothelial carcinoma with an effective amount of infigratinib or a pharmaceutically acceptable salt thereof, comprising:

testing a sample obtained from the patient after administration of the effective amount of infigratinib or a pharmaceutically acceptable salt thereof to measure the allele frequency of at least one FGFR3 biomarker in the patient's cell-free DNA (cfDNA),
wherein detection of the at least one FGFR3 biomarker at a lower variant allele frequency in the patient's cfDNA compared to a baseline allele frequency of the at least one FGFR3 biomarker is indicative of the response of the patient to the treatment, and
wherein the baseline allele frequency measurement is the allele frequency measured in the patient's cfDNA prior to administration of the effective amount of infigratinib or a pharmaceutically acceptable salt thereof.

In another aspect, provided herein are methods of identifying a patient for treatment of an upper tract urothelial carcinoma with an effective amount of infigratinib or a pharmaceutically acceptable salt thereof, comprising:

testing a sample obtained from the patient after administration of the effective amount of infigratinib or a pharmaceutically acceptable salt thereof to measure gene expression of at least one FGFR3 biomarker in the patient's cell-free DNA (cfDNA),
wherein detection of an alteration in the level of expression of the at least one FGFR3 biomarker in the patient's cfDNA compared to a baseline gene expression measurement is indicative of the candidacy of the patient for treatment, and
wherein the baseline gene expression measurement is the gene expression measured in the patient's cfDNA prior to administration of the effective amount of infigratinib or a pharmaceutically acceptable salt thereof.

A method for monitoring the response of a patient to treatment by an effective amount of infigratinib or a pharmaceutically acceptable salt thereof for an upper tract urothelial carcinoma, comprising:

testing a sample obtained from the patient after administration of the effective amount of infigratinib or a pharmaceutically acceptable salt thereof to measure the allele frequency of at least one FGFR3 biomarker in the patient's cell-free DNA (cfDNA),
wherein detection of the at least one FGFR3 biomarker at a lower variant allele frequency in the patient's cfDNA compared to a baseline allele frequency of the at least one FGFR3 biomarker is indicative of the response of the patient to the treatment, and
wherein the baseline allele frequency measurement is the allele frequency measured in the patient's cfDNA prior to administration of the effective amount of infigratinib or a pharmaceutically acceptable salt thereof.

A method for monitoring the response of a patient to treatment by an effective amount of infigratinib or a pharmaceutically acceptable salt thereof for an upper tract urothelial carcinoma, comprising:

testing a sample obtained from the patient after administration of the effective amount of infigratinib or a pharmaceutically acceptable salt thereof to measure gene expression of at least one FGFR3 biomarker in the patient's cell-free DNA (cfDNA),
wherein detection of an alteration in the level of expression of the at least one FGFR3 biomarker in the patient's cfDNA compared to a baseline gene expression measurement is indicative of the response of the patient to the treatment, and
wherein the baseline gene expression measurement is the gene expression measured in the patient's cfDNA prior to administration of the effective amount of infigratinib or a pharmaceutically acceptable salt thereof.

In certain embodiments, the sample obtained from the patient is a blood sample.

In certain embodiments, the at least one FGFR3 biomarker is selected from the group comprising ERK, pERK, STAT, pSTAT, RAF, pRAF, or combinations thereof.

In another aspect, provided herein are methods for treating a urothelial carcinoma in a patient in need thereof. In certain embodiments, the patient has previously had a nephro-ureterectomy, a distal ureterectomy, or a cystectomy.

In certain embodiments, provided herein are methods of treating a urothelial carcinoma in a patient in need thereof, comprising administering to the patient an effective amount of infigratinib, or a pharmaceutically acceptable salt thereof, wherein the patient has previously had a nephro-ureterectomy, a distal ureterectomy, or a cystectomy.

In certain embodiments, provided herein are methods of treating a urothelial carcinoma in a patient in need thereof, comprising administering to the patient an effective amount of infigratinib, or a pharmaceutically acceptable salt thereof, wherein the patient has previously had a nephro-ureterectomy, a distal ureterectomy, or a cystectomy within 120 days of the administering.

In certain embodiments, the urothelial carcinoma is an invasive upper tract urothelial carcinoma or urothelial carcinoma of the bladder. In certain embodiments, the urothelial carcinoma is a non-invasive upper tract urothelial carcinoma or urothelial carcinoma of the bladder.

In certain embodiments, the patient has previously been administered a cisplatin-based chemotherapy but has a residual carcinoma.

In certain embodiments, the urothelial carcinoma is histologically or cytologically confirmed.

In certain embodiments, the urothelial carcinoma has a FGFR1 mutation, gene rearrangement or fusion. In certain embodiments, the urothelial carcinoma has a FGFR1 gene fusion. In some embodiments, the FGFR1 gene fusion comprises a FGFR1 gene fusion partner selected from the group consisting of BAG4, ERLIN2, NTM, FGFR1OP2, TACC3, and TRP. In some embodiments, the FGFR1 gene fusion comprises a FGFR1 gene fusion partner, wherein the FGFR1 gene fusion partner is NTM.

In certain embodiments, the urothelial carcinoma has a FGFR2 mutation, gene rearrangement or fusion. In certain embodiments, the urothelial carcinoma has a FGFR2 gene fusion. In some embodiments, the FGFR2 gene fusion comprises a FGFR2 gene fusion partner selected from the group consisting of 10Q26.13, AFF1, AFF3, AFF4, AHCYL1, ALDH1L2, ARFIP1, BAG4, BAIAP2L1, BICC1, C10orf118, C10orf68, C7, CASC15, CASP7, CCDC147, CCDC6, CELF2, CIT, COL14A1, CREB5, CREM, DNAJC12, ERLIN2, HOOK1, INA, KCTD1, KIAA1217, KIAA1598, KIAA1967, KIFC3, MGEA5, NCALD, NOL4, NPM1, NRAP, OFD1, OPTN, PARK2, PAWR, PCMI, PDHX, PHLDB2, PPAPDC1A, PPHLN1, RASAL2, SFMBT2, SLC45A3, SLMAP, SLMAP2, SORBS1, STK26, STK3, TACC1, TACC2, TACC3, TBC1D1, TEL, TFEC, TRA2B, UBQLN1, VCL, WAC, ZMYM4, and FGFROP2.

In certain embodiments, the urothelial carcinoma has a FGFR3 mutation, gene rearrangement or fusion. In certain embodiments, the urothelial carcinoma has a FGFR3 gene fusion. In some embodiments, the FGFR3 gene fusion comprises a FGFR3 gene fusion partner selected from the group consisting of BAIAP2L1, JAKMIP1, TACC3, TNIP2, and WHSC1. In some embodiments, the FGFR3 gene fusion comprises a FGFR3 gene fusion partner selected from the group consisting of BAIAP2L1, JAKMIP1, TACC3, and TNIP2.

In certain embodiments, the patient undergoes molecular prescreening, for example using next generation sequencing, circulating tumor DNA analysis or a fluorescence in situ hybridization assay, to determine whether the upper tract urothelial carcinoma has a FGFR1, FGFR2, or FGFR3 mutation, gene rearrangement or gene fusion. In some embodiments, the molecular prescreening occurs prior to administration of the effective amount of infigratinib, or a pharmaceutically acceptable salt thereof. In some embodiments, the molecular prescreening to occurs prior to the previous administration of a cisplatin-based chemotherapeutic therapy.

In certain embodiments, the urothelial carcinoma has a FGFR1, FGFR2, and/or FGFR3 mutation. In certain embodiments, the urothelial carcinoma has a FGFR1 mutation. In certain embodiments, the urothelial carcinoma has a FGFR2 mutation. In certain embodiments, the urothelial carcinoma has a FGFR3 mutation.

In certain embodiments, the FGFR1 mutation is selected from the group consisting of FGFR1 G818R, FGFR1 K656E, FGFR1 N546K, FGFR1 R445W, FGFR1 T141R, and combinations thereof.

In certain embodiments, the FGFR2 mutation is selected from the group consisting of FGFR2 D471N, FGFR2 A315T, FGFR2 D336N, FGFR2 P253R, FGFR2 S252W, FGFR2 Y375C, FGFR2 I547V, FGFR2 K659E, FGFR2 N549K, FGFR2 N549S, FGFR2 N549Y, FGFR2 V395D, FGFR2 C382R, FGFR2 E565A, FGFR2 K641R, FGFR2 K659M, FGFR2 L617V, FGFR2 N549H, FGFR2 N550K, FGFR2 V564F, and combinations thereof.

In certain embodiments, the FGFR3 mutation is selected from the group consisting of FGFR3 A391E, FGFR3 A393E, FGFR3 D785Y, FGFR3 E627K, FGFR3 G370C, FGFR3 G372C, FGFR3 G380R, FGFR3 K650E, FGFR3 K652E/Q, FGFR3 K650M, FGFR3 K652M/T, FGFR3 K650N, FGFR3 K650T, FGFR3 K652E, FGFR3 N540S, FGFR3 R248C, FGFR3 R399C, FGFR3 S131L, FGFR3 S249C, FGFR3 S249C, FGFR3 S371C, FGFR3 V555M, FGFR3 V6771, FGFR3 Y373C, FGFR3 Y375C, and combinations thereof. In some embodiments, the FGFR3 mutation is selected from the group consisting of FGFR3 R248C, FGFR3 S249C, FGFR3 G372C, FGFR3 A393E, FGFR3 Y375C, FGFR3 K652M/T, FGFR3 K652E/Q, and combinations thereof. In certain embodiments, the FGFR3 mutations described herein correspond to the amino acid numbering for the FGFR3 mRNA isoform NM_001163213.1. In certain embodiments, the amino acid numbering corresponding to the analogous position in various FGFR3 isoforms may differ (e.g., A393E becomes A391E in isoform NM_00142.5), as would be known to a person of skill in the art.

In certain embodiments, administering an effective amount of infigratinib, or a pharmaceutically acceptable salt thereof, comprises administering orally about 125 mg of infigratinib, or a pharmaceutically acceptable salt thereof, once daily. In certain embodiments, administering an effective amount of infigratinib, or a pharmaceutically acceptable salt thereof, comprises administering orally about 100 mg of infigratinib, or a pharmaceutically acceptable salt thereof, once daily. In certain embodiments, administering an effective amount of infigratinib, or a pharmaceutically acceptable salt thereof, comprises administering orally about 75 mg of infigratinib, or a pharmaceutically acceptable salt thereof, once daily. In certain embodiments, administering an effective amount of infigratinib, or a pharmaceutically acceptable salt thereof, comprises administering orally about 50 mg of infigratinib, or a pharmaceutically acceptable salt thereof, once daily. In certain embodiments, administering an effective amount of infigratinib, or a pharmaceutically acceptable salt thereof, comprises administering orally about 25 mg of infigratinib, or a pharmaceutically acceptable salt thereof, once daily.

In certain embodiments, administering an effective amount of infigratinib, or a pharmaceutically acceptable salt thereof, comprises a 28-day cycle, wherein about 125 mg of infigratinib, or a pharmaceutically acceptable salt thereof, is administered orally once daily to the patient for 3 consecutive weeks, and no infigratinib is administered for 1 week. In certain embodiments, administering an effective amount of infigratinib, or a pharmaceutically acceptable salt thereof, comprises a 28-day cycle, wherein about 100 mg of infigratinib, or a pharmaceutically acceptable salt thereof, is administered orally once daily to the patient for 3 consecutive weeks, and no infigratinib is administered for 1 week. In certain embodiments, administering an effective amount of infigratinib, or a pharmaceutically acceptable salt thereof, comprises a 28-day cycle, wherein about 75 mg of infigratinib, or a pharmaceutically acceptable salt thereof, is administered orally once daily to the patient for 3 consecutive weeks, and no infigratinib is administered for 1 week. In certain embodiments, administering an effective amount of infigratinib, or a pharmaceutically acceptable salt thereof, comprises a 28-day cycle, wherein about 50 mg of infigratinib, or a pharmaceutically acceptable salt thereof, is administered orally once daily to the patient for 3 consecutive weeks, and no infigratinib is administered for 1 week. In certain embodiments, administering an effective amount of infigratinib, or a pharmaceutically acceptable salt thereof, comprises a 28-day cycle, wherein about 25 mg of infigratinib, or a pharmaceutically acceptable salt thereof, is administered orally once daily to the patient for 3 consecutive weeks, and no infigratinib is administered for 1 week.

In certain embodiments, the effective amount of infigratinib, or a pharmaceutically acceptable salt thereof is administered to the patient for two, three, four, five, six, seven, eight, nine, ten, eleven, or twelve consecutive 28-days cycles. In some embodiments, the effective amount of infigratinib, or a pharmaceutically acceptable salt thereof is administered to the patient for two consecutive 28-days cycles.

In certain embodiments, the about 125 mg of infigratinib, or a pharmaceutically acceptable salt thereof, is provided as a unit dose. In certain embodiments, the about 100 mg of infigratinib, or a pharmaceutically acceptable salt thereof, is provided as a unit dose. In certain embodiments, the about 75 mg of infigratinib, or a pharmaceutically acceptable salt thereof, is provided as a unit dose. In certain embodiments, the about 50 mg of infigratinib, or a pharmaceutically acceptable salt thereof, is provided as a unit dose. In certain embodiments, the about 25 mg of infigratinib, or a pharmaceutically acceptable salt thereof, is provided as a unit dose.

In certain embodiments, the about 125 mg of infigratinib, or a pharmaceutically acceptable salt thereof, is provided as a 100 mg unit dose and a 25 mg unit dose. In some embodiments, the about 125 mg of infigratinib, or a pharmaceutically acceptable salt thereof, is provided as a 75 mg unit dose and a 50 mg unit dose.

In certain embodiments, the about 100 mg of infigratinib, or a pharmaceutically acceptable salt thereof, is provided as a 75 mg unit dose and a 25 mg unit dose. In some embodiments, the about 100 mg of infigratinib, or a pharmaceutically acceptable salt thereof, is provided as two 50 mg unit doses.

In certain embodiments, the about 75 mg of infigratinib, or a pharmaceutically acceptable salt thereof, is provided as a 50 mg unit dose and a 25 mg unit dose.

In certain embodiments, the about 50 mg of infigratinib, or a pharmaceutically acceptable salt thereof, is provided as two 25 mg unit doses.

In certain embodiments, the methods include administrating an effective amount of a pharmaceutically acceptable salt of infigratinib to a patient in need thereof. In some embodiments, the pharmaceutically acceptable salt of infigratinib is a monophosphate salt. In some embodiments, the pharmaceutically acceptable salt of infigratinib is an anhydrous monophosphate salt. In some embodiments, the pharmaceutically acceptable salt of infigratinib is an anhydrous monophosphate salt in a polymorphic form characterized by an X-ray powder diffraction (XRPD) peak (2 Theta) at 15.0°±0.2°. In some embodiments, the polymorphic form of the anhydrous crystalline monophosphate salt of infigratinib is described herein.

In another aspect, provided herein are methods of treating an upper tract urothelial carcinoma in a patient in need thereof, comprising administering any one of the pharmaceutical compositions disclosed herein to the patient.

In another aspect, provided herein are methods of treating a urothelial carcinoma in a patient in need thereof, comprising administering any one of the pharmaceutical compositions disclosed herein to the patient, wherein the patient has previously had a nephro-ureterectomy, a distal ureterectomy, or a cystectomy.

In another aspect, provided herein are methods of treating a urothelial carcinoma in a patient in need thereof, comprising administering any one of the pharmaceutical compositions disclosed herein to the patient, wherein the patient has previously had a nephro-ureterectomy, a distal ureterectomy, or a cystectomy. In some embodiments, the patient has previously had a nephron-ureterectomy, a distal ureterectomy, or a cystectomy within 120 days of the administering.

In another aspect, provided herein are methods of treating an upper tract urothelial carcinoma in a patient in need thereof, comprising administering any one of the pharmaceutical compositions disclosed herein to the patient, wherein the pharmaceutical composition is administered as a neoadjuvant therapy.

In another aspect, provided herein are methods of treating a patient in need thereof having an upper tract urothelial carcinoma with at least one FGFR3 mutation, gene rearrangement or gene fusion, the method comprising:
(i) obtaining a sample from the patient;
(ii) analyzing the sample for the presence of the at least one FGFR3 mutation, gene rearrangement or gene fusion; and
(iii) administering any one of the pharmaceutical compositions disclosed herein to the patient,
   wherein the effective amount of infigratinib or a pharmaceutically acceptable salt thereof is administered as a neoadjuvant therapy.

In certain embodiments, the sample is obtained from the patient using a method selected from the group consisting of a selective upper tract washing, fine needle aspirates, core needle biopsy, brush biopsy, urine cell free DNA, blood cell free DNA, and other cytology samples (for cytology sampling of metastatic sites such as pleural effusions, etc.).

In another aspect, provided herein are methods of treating a patient in need thereof having an upper tract urothelial carcinoma with at least one FGFR3 mutation, gene rearrangement or gene fusion, the method comprising:
(i) obtaining a sample from the patient, wherein the sample is obtained from the patient using a selective upper tract washing;
(ii) analyzing the sample for the presence of the at least one FGFR3 mutation, gene rearrangement or gene fusion; and
(iii) administering any one of the pharmaceutical compositions disclosed herein to the patient,
   wherein the effective amount of infigratinib or a pharmaceutically acceptable salt thereof is administered as a neoadjuvant therapy.

### Treatment of Non-Muscle Invasive Bladder Cancer

In one aspect, provided herein are methods of treating non-muscle invasive bladder cancer in a patient in need thereof. Non-muscle invasive bladder cancer may also be referred to as intermediate-risk non-muscle invasive bladder cancer or high-grade non-invasive papillary urothelial carcinoma.

In certain embodiments, provided herein are methods of treating non-muscle invasive bladder cancer in a patient in need thereof, comprising administering to the patient an effective amount of infigratinib, or a pharmaceutically acceptable salt thereof.

In certain embodiments, the patient has reoccurrence of the non-muscle invasive bladder cancer after previous administration of another therapy.

In certain embodiments, provided herein are methods of treating non-muscle invasive bladder cancer in a patient in need thereof, comprising administering an effective amount of infigratinib, or a pharmaceutically acceptable salt thereof, wherein the patient has reoccurrence of the non-muscle invasive bladder cancer after previous administration of another therapy.

In certain embodiments, the previous administration of another therapy is a therapy for non-muscle invasive bladder cancer. In some embodiments, the previous administration of another therapy is an administration of an immunotherapeutic agent. In some embodiments, the previous administration of an immunotherapeutic agent is a bacillus Calmette-Guerin-containing regimen.

In certain embodiments, the non-muscle invasive bladder cancer has a FGFR3 mutation, gene rearrangement or gene fusion.

In certain embodiments, the non-muscle invasive bladder cancer has a FGFR3 mutation. In some embodiments, the FGFR3 mutation is selected from the group consisting of FGFR3 K650E, FGFR3 S249C, FGFR3 R248C, FGFR3 Y375C, FGFR3 G372C, FGFR3 S373C, FGFR3 A393E, FGFR3 A371A, FGFR3 I378C, FGFR3 L379L, FGFR3 G382R, and combinations thereof.

In certain embodiments, the non-muscle invasive bladder cancer has a FGFR3 gene fusion. In some embodiments, the FGFR3 gene fusion comprises the FGFR3 gene fusion partner TACC3.

In certain embodiments, administering an effective amount of infigratinib, or a pharmaceutically acceptable salt thereof, comprises administering about 125 mg of infigratinib, or a pharmaceutically acceptable salt thereof, once daily. In certain embodiments, administering an effective amount of infigratinib, or a pharmaceutically acceptable salt thereof, comprises administering about 100 mg of infigratinib, or a pharmaceutically acceptable salt thereof, once daily. In certain embodiments, administering an effective amount of infigratinib, or a pharmaceutically acceptable salt thereof, comprises administering about 75 mg of infigratinib, or a pharmaceutically acceptable salt thereof, once daily. In certain embodiments, administering an effective amount of infigratinib, or a pharmaceutically acceptable salt thereof, comprises administering about 50 mg of infigratinib, or a pharmaceutically acceptable salt thereof, once daily. In certain embodiments, administering an effective amount of infigratinib, or a pharmaceutically acceptable salt thereof, comprises administering about 25 mg of infigratinib, or a pharmaceutically acceptable salt thereof, once daily.

In certain embodiments, administering an effective amount of infigratinib, or a pharmaceutically acceptable salt thereof comprises a 28-day cycle, wherein about 125 mg of infigratinib, or a pharmaceutically acceptable salt thereof, is administered once daily to the patient for 3 consecutive weeks, and no infigratinib is administered for 1 week. administering an effective amount of infigratinib, or a pharmaceutically acceptable salt thereof comprises a 28-day cycle, wherein about 100 mg of infigratinib, or a pharmaceutically acceptable salt thereof, is administered once daily to the patient for 3 consecutive weeks, and no infigratinib is administered for 1 week. administering an effective amount of infigratinib, or a pharmaceutically acceptable salt thereof comprises a 28-day cycle, wherein about 75 mg of infigratinib, or a pharmaceutically acceptable salt thereof, is administered once daily to the patient for 3 consecutive weeks, and no infigratinib is administered for 1 week. administering an effective amount of infigratinib, or a pharmaceutically acceptable salt thereof comprises a 28-day cycle, wherein about 50 mg of infigratinib, or a pharmaceutically acceptable salt thereof, is administered once daily to the patient for 3 consecutive weeks, and no infigratinib is administered for 1 week. administering an effective amount of infigratinib, or a pharmaceutically acceptable salt thereof comprises a 28-day cycle, wherein about 25 mg of infigratinib, or a pharmaceutically acceptable salt thereof, is administered once daily to the patient for 3 consecutive weeks, and no infigratinib is administered for 1 week.

In certain embodiments, the about 125 mg of infigratinib, or a pharmaceutically acceptable salt thereof, is provided as a unit dose. In certain embodiments, the about 100 mg of infigratinib, or a pharmaceutically acceptable salt thereof, is provided as a unit dose. In certain embodiments, the about 75 mg of infigratinib, or a pharmaceutically acceptable salt thereof, is provided as a unit dose. In certain embodiments, the about 50 mg of infigratinib, or a pharmaceutically acceptable salt thereof, is provided as a unit dose. In certain embodiments, the about 25 mg of infigratinib, or a pharmaceutically acceptable salt thereof, is provided as a unit dose.

In certain embodiments, the about 125 mg of infigratinib, or a pharmaceutically acceptable salt thereof, is provided as a 100 mg unit dose and a 25 mg unit dose. In some embodiments, the about 125 mg of infigratinib, or a pharmaceutically acceptable salt thereof, is provided as a 75 mg unit dose and a 50 mg unit dose.

In certain embodiments, the about 100 mg of infigratinib, or a pharmaceutically acceptable salt thereof, is provided as a 75 mg unit dose and a 25 mg unit dose. In some embodiments, the about 100 mg of infigratinib, or a pharmaceutically acceptable salt thereof, is provided as two 50 mg unit doses.

In certain embodiments, the about 75 mg of infigratinib, or a pharmaceutically acceptable salt thereof, is provided as a 50 mg unit dose and a 25 mg unit dose.

In certain embodiments, the about 50 mg of infigratinib, or a pharmaceutically acceptable salt thereof, is provided as two 25 mg unit doses.

In certain embodiments, the about 125 mg of infigratinib or a pharmaceutically acceptable salt thereof is administered orally to the patient. In certain embodiments, the about 100 mg of infigratinib or a pharmaceutically acceptable salt thereof is administered orally to the patient. In certain embodiments, the about 75 mg of infigratinib or a pharmaceutically acceptable salt thereof is administered orally to the patient. In certain embodiments, the about 50 mg of infigratinib or a pharmaceutically acceptable salt thereof is administered orally to the patient. In certain embodiments, the about 25 mg of infigratinib or a pharmaceutically acceptable salt thereof is administered orally to the patient.

In certain embodiments, the effective amount of infigratinib or a pharmaceutically acceptable salt thereof is administered to the patient via local administration. In certain embodiments, the effective amount of infigratinib or a pharmaceutically acceptable salt thereof is administered to the patient intravesically. In certain embodiments, the effective amount of infigratinib or a pharmaceutically acceptable salt thereof is administered to the patient intratumorally.

In certain embodiments, the effective amount of infigratinib or a pharmaceutically acceptable salt thereof is delivered via insertion of a controlled release, implantable device into the patient's bladder. In certain embodiments, the effective amount of infigratinib or a pharmaceutically acceptable salt thereof is delivered via insertion of a controlled release, implantable device into the patient's ureter. In certain embodiments, the effective amount of infigratinib or a pharmaceutically acceptable salt thereof is delivered via insertion of a controlled release, implantable device into the patient's renal pelvis.

In certain embodiments, the controlled release, implantable device is a dual-lumen silicon tube comprising a superelastic wireform. An example of a dual-lumen silicon tube comprising a superelastic wireform is a Taris device.

In certain embodiments, the controlled release, implantable device is a gel. In some embodiments, the gel is a biodegradable gel. An example of a biodegradable gel is a hydrogel.

In certain embodiments, the methods include administrating an effective amount of a pharmaceutically acceptable salt of infigratinib to a patient in need thereof. In some embodiments, the pharmaceutically acceptable salt of infigratinib is a monophosphate salt. In some embodiments, the pharmaceutically acceptable salt of infigratinib is an anhydrous monophosphate salt. In some embodiments, the pharmaceutically acceptable salt of infigratinib is an anhydrous monophosphate salt in a polymorphic form characterized by an X-ray powder diffraction (XRPD) peak (2 Theta) at 15.0°±0.2°. In some embodiments, the polymorphic form of the anhydrous crystalline monophosphate salt of infigratinib is described herein.

In certain embodiments, the methods provided herein further comprise administering an effective amount of a second therapeutic agent to the patient. In certain embodiments, the effective amount of the second therapeutic agent is administered to the patient via local administration. In certain embodiments, the effective amount of the second therapeutic agent is administered to the patient intravesically.

In certain embodiments, the effective amount of the second therapeutic agent is delivered via insertion of a controlled release, implantable device into the patient's bladder. In certain embodiments, the effective amount of the second therapeutic agent is delivered via insertion of a controlled release, implantable device into the patient's ureter. In certain embodiments, the effective amount of the second therapeutic agent is delivered via insertion of a controlled release, implantable device into the patient's renal pelvis.

In certain embodiments, the controlled release, implantable device is a dual-lumen silicon tube comprising a superelastic wireform. An example of a dual-lumen silicon tube comprising a superelastic wireform is a Taris device.

In certain embodiments, the controlled release, implantable device is a gel. In some embodiments, the gel is a biodegradable gel. An example of a biodegradable gel is a hydrogel.

In certain embodiments, the second therapeutic agent is gemcitabine or a pharmaceutically acceptable salt thereof.

In another aspect, provided herein are methods of treating non-muscle invasive bladder cancer in a patient in need thereof, comprising administering any one of the pharmaceutical compositions disclosed herein to the patient.

In another aspect, provided herein are methods of treating non-muscle invasive bladder cancer in a patient in need thereof, comprising administering any one of the pharmaceutical compositions disclosed herein to the patient, wherein the patient has reoccurrence of the non-muscle invasive bladder cancer after previous administration of another therapy.

### EXAMPLES

In order that the disclosure described herein may be more fully understood, the following examples are set forth. The synthetic and biological examples described in this application are offered to illustrate the compounds, pharmaceutical compositions, and methods provided herein and are not to be construed in any way as limiting their scope.

### Example 1: Synthesis of 3-(2,6-dichloro-3,5-dimethoxy-phenyl)-1-{6-4-(4-ethyl-piperazin-1-yl)-phenylaminol-pyrimidin-4-yl}-1-methyl-urea (infigratinib)

### Step A: Synthesis of N-4-(4-ethyl-piperazin-1-yl)-phenyl)-N′-methyl-pyrimidine-4,6-diamine

A mixture of 4-(4-ethylpiperazin-1-yl)-aniline (1 g, 4.88 mmol), (6-chloro-pyrimidin-4-yl)-methyl-amine (1.81 g, 12.68 mmol. 1.3 eq.), and 4N HCl in dioxane (15 mL) is heated in a sealed tube to 150° C. for 5 hours. The reaction mixture is concentrated, diluted with dichloromethane (DCM) and a saturated aqueous solution of sodium bicarbonate. The aqueous layer is separated and extracted with DCM. The organic phase is washed with brine, dried (sodium sulfate), filtered and concentrated. Purification of the residue by silica gel column chromatography (DCM/MeOH, 93:7) followed by trituration in diethyl ether affords the title compound as a white solid: ESI-MS: 313.2 [MH]⁺; t_{R}=1.10 min (gradient J); TLC: R_{f}=0.21 (DCM/MeOH, 93:7).

### Step B: Synthesis of 4-(4-ethylpiperazin-1-yl)-aniline

A suspension of 1-ethyl-4-(4-nitro-phenyl)-piperazine (6.2 g, 26.35 mmol) and Raney Nickel (2 g) in MeOH (120 mL) is stirred for 7 hours at RT, under a hydrogen atmosphere. The reaction mixture is filtered through a pad of celite and concentrated to afford 5.3 g of the title compound as a violet solid: ESI-MS: 206.1 [MH]⁺; TLC: R_{f}=0.15 (DCM/MeOH+1% NH₃^{aq}, 9:1).

### Step C: Synthesis of 1-ethyl-4-(4-nitro-phenyl)-piperazine

A mixture of 1-bromo-4-nitrobenzene (6 g, 29.7 mmol) and 1-ethylpiperazine (7.6 mL, 59.4 mmol, 2 eq.) is heated to 80° C. for 15 hours. After cooling to RT, the reaction mixture is diluted with water and DCM/MeOH, 9:1. The aqueous layer is separated and extracted with DCM/MeOH, 9:1. The organic phase is washed with brine, dried (sodium sulfate), filtered and concentrated. Purification of the residue by silica gel column chromatography (DCM/MeOH+1% NH₃^{aq}, 9:1) affords 6.2 g of the title compound as a yellow solid: ESI-MS: 236.0 [MH]⁺; t_{R}=2.35 min (purity: 100%, gradient J); TLC: R_{f}=0.50 (DCM/MeOH+1% NH₃^{aq}, 9:1).

### Step D: Synthesis of (6-chloro-pyrimidin-4-yl)-methyl-amine

This material was prepared by a modified procedure published in the literature (J. Appl. Chem. 1955, 5, 358): To a suspension of commercially available 4,6-dichloropyrimidine (20 g, 131.6 mmol, 1.0 eq.) in isopropanol (60 mL) is added 33% methylamine in ethanol (40.1 mL, 328.9 mmol, 2.5 eq.) at such a rate that the internal temperature does not rise above 50° C. After completion of the addition the reaction mixture was stirred for 1 hour at room temperature. Then, water (50 mL) is added and the suspension formed is chilled in an ice bath to 5° C. The precipitated product is filtered off, washed with cold isopropanol/water 2:1 (45 mL) and water. The collected material is vacuum dried over night at 45° C. to afford the title compound as colorless powder: t_{R}=3.57 min (purity: >99%, gradient A), ESI-MS: 144.3/146.2 [MH]⁺.

### Step E: Synthesis of 3-(2,6-dichloro-3,5-dimethoxy-phenyl)-1-{6-4-(4-ethyl-piperazin-1-yl)-phenylaminol-pyrimidin-4-yl}-1-methyl-urea

The title compound was prepared by adding 2,6-dichloro-3,5-dimethoxyphenyl-isocyanate (1.25 eq.) to a solution of N-4-(4-ethyl-piperazin-1-yl)-phenyl)-N′-methyl-pyrimidine-4,6-diamine (2.39 g, 7.7 mmol, 1 eq.) in toluene and stirring the reaction mixture for 1.5 hours at reflux. Purification of the crude product by silica gel column chromatography (DCM/MeOH+1% NH₃^{aq}, 95:5) affords the title compound as a white solid: ESI-MS: 560.0/561.9 [MH]⁺; t_{R}=3.54 min (purity: 100%, gradient J); TLC: R_{f}=0.28 (DCM/MeOH+1% NH₃^{aq}, 95:5). Analysis: C₂₆H₃₁N₇O₃Cl₂, calc. C, 55.72%; H, 5.57%; N, 17.49%; O, 8.56%; Cl, 12.65%. Found C, 55.96%: H, 5.84%: N, 17.17%; O, 8.46%; Cl, 12.57%.

### Example 2: Synthesis of the Monophosphate Salt Form A of 3-(2,6-dichloro-3,5-dimethoxy-phenyl)-1-{6-4-(4-ethyl-piperazin-1-yl)-phenylaminol-pyrimidin-4-yl}-1-methyl-urea (BGJ398)

To a round bottom flask was added 3-(2,6-dichloro-3,5-dimethoxyphenyl)-1-(6-4-(4-ethylpiperazin-1-yl)phenylaminol-pyrimidine-4-yl)-1-methyl-urea (134 g, 240 mmol) and isopropanol (IPA) (2000 mL). The suspension was stirred and heated to 50° C. and a solution of phosphoric acid (73.5 g, 750 mmol) in water (2000 mL) added to it portions. The mixture was stirred at 60° C. for 30 minutes and filtered through a polypropylene pad. The pad was washed with warm IPA/water (1:1, 200 mL) and the filtrates were combined. To this clear solution, IPA (6000 mL) was added and the mixture was stirred under reflux for 20 minutes, cooled slowly to room temperature (25° C.), and stirred for 24 hours. The white salt product was collected by filtration, washed with IPA (2×500 mL) and dried in the oven at 60° C. under reduced pressure for two days to provide the anhydrous crystalline monophosphate salt (110 g). Yield 70%. Purity>98% by HPLC. Analysis: C₂₆H₃₄N₇O₇Cl₂P, calc. C, 47.42%; H, 5.20%; N, 14.89%; O, 17.01%; Cl, 10.77%; P. 4.70%. Found C, 47.40%; H, 5.11%: N, 14.71%; O, 17.18%: Cl, 10.73%; P 4.87%.

### Example 3: Manufacturing Process for 25 mg, 100 mg, and 125 mg Dose Infigratinib Pharmaceutical Formulations

In the following example, the manufacturing process is outlined for all exemplified dosage strengths.

The corresponding amounts of the ingredients are provided in the formulas under Examples 3.1, 3.2, and 3.3 below.

### Manufacture of the Pharmaceutical Blend

Cellulose MK-GR, lactose (milled), infigratinib, cellulose HPM 603 and cross-linked polyvinylpyrrolidone (PVP-XL) are sequentially added into a vertical wet high-shear granulator (e.g., TK Fiedler (bottom driven, 65 L) with a granulator fill volume of about 45-50%, the five components are then mixed at an impeller setting of 60-270 rpm, preferably 150 rpm; and a chopper setting of 600-3000 rpm, preferably 1500 rpm, for about 5 min to obtain a dry blend.

Purified water is added as granulation liquid at rate of about 385 g/min for 7 min (adding up to about 2.7 kg of water) with a spray setting pressure of 1.5 bar (impeller setting of 60-270 rpm, preferably 150 rpm; and chopper settings of 600-3000 rpm, preferably 1500 rpm). The resulting granulation mixture is kneaded for about 3 min (impeller setting of 60-270 rpm, preferably 150 rpm; and chopper setting of 600-3000 rpm, preferably 1500 rpm). The kneaded granulation mass is screened through a 3.0 mm sieve using a Comil with 90-600 rpm. This process step is optional and may be omitted, but preferably this process step is performed.

The granules are dried in a fluidized bed dryer, e.g., Glatt GPCG 15/30 or equivalent, with an inlet air temperature of 55-65° C., preferably 60° C., a product temperature of about 30-40° C. and an inlet air volume of 300-1200 m³/h to reach a drying endpoint of ≤2.2%.

The dried granules are screened through 800-1000 μm in a Comil. The resulting dried and screened granules are also referred to herein as an inner phase.

The outer phase excipients PVP XL and Aerosil 200 are screened through 900-1000 μm in a Comil with ca. 50-150 rpm and then combined with the inner phase in a suitable container (e.g., bin blender, turbula or equivalent) by mixing with 4-25 rpm, preferably 17 rpm for about 5 min (33-66% powder fill).

The solids are lubricated by the addition of 500 rpm screened magnesium stearate as an additional outer phase excipient by blending in a diffusion mixer (tumble) or bin blender (e.g., Bohle PM400, Turbula, or equivalent) for about 3 min at about 17 rpm, to obtain the final blend which is ready for capsule filling.

### Manufacturing of the Capsule

The final blend is then filled into hard gelatin capsules (HGC) of size 0, 1, or 3 by encapsulation machines with dosing plate principle or with dosing tube (e.g., Höfliger & Karg GKF 330, Bosch GKF 1500, Zanasi 12 E. Zanasi 40 E) with encapsulation speeds of 10,000 up to 100,000 caps/h and without precompression. The weights of the capsules are controlled and the capsules dedusted.

### Example 3.1

TABLE 1
Formula for 25 mg Dosage Strength

| | Composition | Composition | Quantity per |
|---|---|---|---|
| | per unit | per Unit | 173’016 units |
| Component | [%] | [mg/unit] | [kg/batch] |
| Infigratinib as | 37.18^{a} | 29.38^{a} | 5.084^{a} |
| monophosphate^{a} | | | |
| Cellulose MK-GR | 25.63 | 20.25 | 3.505 |
| Lactose milled | 29.43 | 23.25 | 4.024 |
| Cellulose HPM603 | 3.16 | 2.50 | 0.433 |
| Polyvinylpolypyrrolidon | 3.16 | 2.50 | 0.433 |
| XL | | | |
| Purified water^{b} | | | |
| Total inner phase | | 77.88 mg | 13.48 kg |
| Polyvinylpolypyrrolidon | 0.10 | 0.08 | 0.0138 |
| XL | | | |
| Aerosil 200 | 0.13 | 0.10 | 0.0177 |
| Magnesium Stearate | 1.20 | 0.95 | 0.164 |
| Total final blend | 100% | 79.01 mg | 13.67 kg |
| Hard gelatin capsule, | | 48.00 mg | |
| size 3 | | | |
| Total capsule weight | | 127.01 mg | |

^{a}The salt factor is 1.175. The drug substance quantity has to be adjusted if the content is ≤99.5%. Respective compensation is done by adjusting lactose content.
^{b}The water used during granulation is removed in the process of drying.

### Example 3.2

TABLE 2
Formula for 100 mg Dosage Strength

| | Composition | Composition | Quantity per |
|---|---|---|---|
| | per unit | per Unit | 173’016 units |
| Component | [%] | [mg/unit] | [kg/batch] |
| Infigratinib as | 37.18^{a} | 117.5^{a} | 5.084^{a} |
| monophosphate^{a} | | | |
| Cellulose MK-GR | 25.63 | 81.0 | 3.505 |
| Lactose milled | 29.43 | 93.0 | 4.024 |
| Cellulose HPM603 | 3.16 | 10.0 | 0.433 |
| Polyvinylpolypyrrolidon | 3.16 | 10.0 | 0.433 |
| XL | | | |
| Purified water^{b} | | | |
| Total inner phase | | 311.5 mg | 13.48 kg |
| Polyvinylpolypyrrolidon | 0.10 | 0.32 | 0.0138 |
| XL | | | |
| Aerosil 200 | 0.13 | 0.41 | 0.0177 |
| Magnesium Stearate | 1.20 | 3.80 | 0.164 |
| Total final blend | 100% | 316.03 mg | 13.67 kg |
| Hard gelatin capsule, | | 76.00 mg | |
| size 1 | | | |
| Total capsule weight | | 392.0 mg | |

^{a}The salt factor is 1.175. The drug substance quantity has to be adjusted if the content is ≤99.5%. Respective compensation is done by adjusting lactose content.
^{b}The water used during granulation is removed in the process of drying.

### Example 3.3

TABLE 3
Formula for 125 mg Dosage Strength

| | Composition | Composition | Quantity per |
|---|---|---|---|
| | per unit | per Unit | 173’016 units |
| Component | [%] | [mg/unit] | [kg/batch] |
| Infigratinib as | 37.18^{a} | 146.875^{a} | 5.084^{a} |
| monophosphate^{a} | | | |
| Cellulose MK-GR | 25.63 | 101.25 | 3.505 |
| Lactose milled | 29.43 | 116.25 | 4.024 |
| Cellulose HPM603 | 3.16 | 12.5 | 0.433 |
| Polyvinylpolypyrrolidon | 3.16 | 12.5 | 0.433 |
| XL | | | |
| Purified water^{b} | | | |
| Total inner phase | | 389.4 mg | 13.48 kg |
| Polyvinylpolypyrrolidon | 0.10 | 0.40 | 0.0138 |
| XL | | | |
| Aerosil 200 | 0.13 | 0.513 | 0.0177 |
| Magnesium Stearate | 1.20 | 4.75 | 0.164 |
| Total final blend | 100% | 395.03 mg | 13.67 kg |
| Hard gelatin capsule, | | 96.00 mg | |
| size 0 | | | |
| Total capsule weight | | 491.0 mg | |

^{a}The salt factor is 1.175. The drug substance quantity has to be adjusted if the content is ≤99.5%. Respective compensation is done by adjusting lactose content.
^{b}The water used during granulation is removed in the process of drying.

### Example 4: A Study of Oral Infigratinib (BGJ398) for the Treatment of Patients with Invasive Urothelial Carcinoma with FGFR3 Genomic Alterations

### Objectives of Study

Primary objective: comparing the centrally reviewed disease-free survival (DFS) of subjects with invasive urothelial carcinoma with FGFR3 genomic alterations treated with infigratinib vs placebo following nephro-ureterectomy, distal ureterectomy, or cystectomy.

Secondary objective: comparing DFS including intraluminal low-risk recurrence in subjects treated with infigratinib vs placebo.

Comparing metastasis-free survival (MFS) of subjects treated with infigratinib vs placebo.

Comparing the overall survival (OS) in subjects treated with infigratinib vs placebo.

To compare investigator-reviewed DFS in subjects treated with infigratinib vs placebo.

Characterize the safety and tolerability of infigratinib when administered as postoperative adjuvant monotherapy.

Compare quality of life (QOL) in subjects treated with infigratinib vs placebo.

Evaluate the PK of infigratinib.

Evaluate cell-free DNA (cfDNA) and/or RNA for mechanisms of resistance.

### Endpoints of Study

Centrally reviewed DFS, from date of randomization to local/regional or contralateral invasive or metastatic recurrence, or death due to any cause, whichever occurs earlier.

Investigator-reviewed DFS including intraluminal low-risk recurrence, from date of randomization to any recurrence or death due to any cause, whichever occurs earlier.

Investigator-reviewed MFS, defined as time from randomization to any metastatic recurrence or death due to any cause, whichever occurs earlier.

OS, defined as time from randomization to death.

Type, frequency, and severity of adverse events (AEs) and serious AEs (SAES), laboratory abnormalities, and other safety findings.

Investigator-reviewed DFS, from date of randomization to local/regional or contralateral invasive or metastatic recurrence, or death due to any cause, whichever occurs earlier.

QOL as measured by the EuroQOL five dimensions questionnaire (EQ-5D) and the European Organization for Research and Treatment of Cancer (EORTC) quality of life questionnaire (QLQ) C30.

Pharmacokinetic (PK) parameter (trough and maximum plasma concentration).

FGFR3 alterations detected by cfDNA and/or RNA sequencing as biomarkers of disease recurrence.

### Study Design

This is a Phase 3 multicenter, double-blind, randomized, placebo-controlled study to evaluate the efficacy of infigratinib in approximately 218 adult subjects with invasive urothelial carcinoma with FGFR3 genomic alterations who are within 120 days following nephro-ureterectomy, distal ureterectomy, or cystectomy and ineligible for cisplatin-based (neo)adjuvant chemotherapy or with residual disease after neoadjuvant therapy. The sample size may be further increased up to a total of 328 subjects based on interim analysis results using an adaptive design promising zone approach. Subjects with invasive urothelial carcinoma include subjects with invasive upper tract urothelial carcinoma (UTUC) and urothelial carcinoma of the bladder (UCB).

Subjects are randomized 1:1 to receive oral infigratinib or placebo administered once daily for the first 3 weeks (21 days) of each 28-day cycle for a maximum of 52 weeks or until local/regional or contralateral invasive or metastatic recurrence, unacceptable toxicity, withdrawal of informed consent, or death. Subjects are evaluated for tumor recurrence radiographically and by urine cytology. For subjects with UTUC (i.e., subjects with a bladder), cystoscopy is performed. Radiography, urine cytology, and cystoscopy continue until metastatic recurrence by blinded independent central review (BICR) or metastatic recurrence by investigator assessment if local/regional or contralateral invasive recurrence by BICR has already occurred. After that time, subjects are followed up for survival status and use of anticancer therapy for 1 year after the final DFS event goal is reached (i.e., end of study).

An interim analysis is conducted after approximately 35 centrally reviewed DFS events have occurred. Based on the results of the interim analysis on DFS, if a sample size increase is deemed necessary using the promising zone approach, the sample size/DFS event goal is increased by a maximum of 50% (328/105). If sample size is increased and event goal is adjusted, then the subsequent analyses are adjusted accordingly time-wise when the adjusted event goal is reached. The details of the sample size adaptation method are pre-specified in the adaptation plan.

Subjects are stratified according to lymph node involvement (yes vs no), prior neoadjuvant chemotherapy (yes vs no), stage (pT2 vs >pT2), and disease (UTUC vs UCB).

Number of subjects: Approximately 218 subjects are initially planned for study participation. The sample size may be increased up to a total of 328 subjects based on interim analysis results using an adaptive design promising zone approach. No more than 15% of the population is enrolled with UCB and no more than 25% of UTUC subjects have pT2 UTUC (limit will be based on stratification).

### Diagnosis and Criteria for Inclusion

Eligible subjects meet all of the following criteria:
1. Are ≥18 years of age of either sex.
2. Have signed informed consent.
3. Have histologically or cytologically confirmed, invasive urothelial carcinoma with susceptible FGFR3 alterations within 120 days following nephro-ureterectomy, distal ureterectomy, or cystectomy.
   Regarding samples and documentation of FGFR3 alterations: FGFR3 mutation is confirmed if: FGFR3 gene is mutated in Exon 7 (R248C, S249C), Exon 10 (G370C, A391E, Y373C), or Exon 15 (K650M/T, K650E/Q); FGFR3 gene fusion or translocation is confirmed if: gene fusion or translocation is identified; the amino acid numbers for the FGFR3 mutations refer to the functional FGFR3 isoform 1 (NP 000133.1) that is the NCBI Refseq ID used to report genetic alterations in FGFR3 by the FoundationOne CDx test; written documentation of central laboratory determination by FoundationOne CDx testing (through Foundation Medicine USA) of FGFR3 alterations is required for study eligibility in study centers outside of China. For study centers in China, confirmation is needed by a test equivalent to that of the central test; for patients who require molecular prescreening to confirm the presence of the FGFR3 alteration to meet the inclusion criteria, an archival tumor sample with a pathology report must be sent to Foundation Medicine USA for FoundationOne CDx testing; for study sites in China, written documentation of FGFR3 alterations by the contracted central laboratory is required for study eligibility.
   If status post neoadjuvant chemotherapy, pathologic stage at surgical resection must be AJCC Stage ≥ypT2 and/or yN+. Prior neoadjuvant therapy is defined as at least 3 cycles of neoadjuvant cisplatin-based chemotherapy with a planned cisplatin dose of 70 mg/m²/cycle. Patients who received less than this or non-cisplatin-based neoadjuvant treatment will be considered as having received no neoadjuvant chemotherapy.
   If not status post neoadjuvant chemotherapy, is ineligible to receive cisplatin-based adjuvant chemotherapy based on Galsky et al. (2011): creatinine clearance ≤60 mL/minute, and/or common Terminology Criteria for Adverse Events (CTCAE version 5.0 or later) Grade ≥2 hearing loss, or CTCAE Grade ≥2 neuropathy.
   If cisplatin ineligible based on Galsky et al. (2011), must also meet the following criteria: upper tract disease should be AJCC Stage ≥pT2 pN0-2 MO (post-lymphadenectomy or no lymphadenectomy [pNx]); UBC should be AJCC Stage ≥pT3 or pN+.
   Must have a centrally reviewed negative postoperative CT (defined as lymph nodes with short axis <1.0 cm and without growth and no distant metastases according to Response Evaluation Criteria in Solid Tumors [RECIST] v1.1) or negative biopsy within 28 days before randomization to confirm absence of disease at baseline.
4. If have had adverse events (AEs) associated with prior surgery or neoadjuvant chemotherapy, they have stabilized or resolved to Grade ≤2 before randomization.
5. Have Eastern Cooperative Oncology Group (ECOG) performance status of ≤2.
6. If a woman of childbearing potential (WOCBP), must have a negative pregnancy test within 7 days of the first dose of study drug. WOCBP and males whose sexual partners are WOCBP must agree to use barrier contraception and a second form of contraception (Clinical Trials Facilitation Group 2014) while receiving study drug and for 3 months following their last dose of study drug. Sexually active males must use a condom during intercourse while taking study drug and for 3 months after the last dose of study drug and should not father a child during this period. Study subjects must agree to refrain from donating sperm and eggs during the study and for 3 months following their last dose of study drug.
7. Are willing and able to comply with study visits and study procedures.

### Exclusion Criteria

To be eligible for the study, a subject must not meet any of the following criteria:
1. Presence of positive surgical margins following nephroureterectomy, distal ureterectomy, or cystectomy.
2. Have received Bacillus Calmette-Guerin (BCG) or other intravesical therapy for NMIBC within the previous 30 days.
3. Are currently receiving or are planning to receive during participation in this study, treatment with agents that are known strong inducers or inhibitors of CYP3A4 and medications which increase serum phosphorus and/or calcium concentration. Subjects are not permitted to receive enzyme-inducing anti-epileptic drugs, including carbamazepine, phenytoin, phenobarbital, and primidone. Prior neoadjuvant chemotherapy or immunotherapy is allowed if inclusion criterion #3 is met. Prior chemotherapy must have been completed with a period of time that is greater than the cycle length used for that treatment prior to first dose of study drug. Subjects who received biologic therapy should have completed therapy with a period that is ≥5 half-lives before the first dose of study drug.
4. Are planning to receive other systemic therapies intended to treat invasive urothelial carcinoma while on this study.
5. Have previously or currently is receiving treatment with a mitogen-activated protein kinase (MEK) or selective FGFR inhibitor.
6. Have a history of primary malignancy within the past 3 years other than (1) invasive UBC or UTUC (i.e., disease under study), (2) noninvasive urothelial carcinoma, (3) any adequately treated in situ carcinoma or non-melanoma carcinoma of the skin, (4) any other curatively treated malignancy that is not expected to require treatment for recurrence during participation in the study, or (5) an untreated cancer on surveillance that may not affect the subject's survival status for years based on clinician assessment/statement. For any other cancers that do not meet the criteria above, written approval is required by the Medical Monitor.
7. Have current evidence of corneal or retinal disorder/keratopathy including, but not limited to, bullous/band keratopathy, inflammation or ulceration, keratoconjunctivitis, confirmed by ophthalmic examination. Subjects with asymptomatic ophthalmic conditions assessed by the Investigator to pose minimal risk for study participation may be enrolled in the study.
8. Have a history and/or current evidence of extensive tissue calcification including, but not limited to, the soft tissue, kidneys, intestine, vasculature, myocardium, and lung with the exception of calcified lymph nodes, minor pulmonary parenchymal calcifications, small renal cyst or stone calcifications, and asymptomatic coronary calcification.
9. Have impaired gastrointestinal (GI) function or GI disease that may significantly alter the absorption of oral infigratinib (e.g., active ulcerative diseases, uncontrolled nausea, vomiting, diarrhea, malabsorption syndrome, small bowel resection).
10. Have current evidence of endocrine alterations of calcium/phosphate homeostasis (e.g., parathyroid disorders, history of parathyroidectomy, tumor lysis, tumoral calcinosis), unless well controlled.
11. Have consumed grapefruit, grapefruit juice, grapefruit hybrids, pomegranates, star fruits, pomelos, or Seville oranges or products containing juice of these fruits within 7 days before the first dose of study drug.
12. Have used medications that are known to prolong the QT interval and/or are associated with a risk of Torsades de Pointes (TdP) within 7 days before the first dose of study drug.
13. Have used amiodarone within 90 days before the first dose of study drug.
14. Have insufficient bone marrow function: absolute neutrophil count (ANC)<1,000/mm³ (1.0×10⁹/L); platelets <75,000/mm3 (<75×109/L); hemoglobin <9.0 g/dL.
15. Have insufficient hepatic and renal function: total bilirubin >1.5× upper limit of normal (ULN) of the testing laboratory (for patients with documented Gilbert syndrome, exclusion for direct bilirubin >1.5×ULN and enrollment requires approval by the Medical Monitor); aspartate aminotransferase/serum glutamic-oxaloacetic transaminase (AST/SGOT) and alanine aminotransferase/serum glutamic-pyruvic transaminase (ALT/SGPT) >2.5×ULN of the testing laboratory; calculated (using the Cockcroft-Gault [C-G] formula (Cockcroft and Gault, 1976) or measured creatinine clearance of <30 mL/min.
16. Have amylase or lipase >2.0×ULN.
17. Have abnormal calcium-phosphate homeostasis: inorganic phosphorus higher than ULN of the testing laboratory; total serum calcium (can be corrected) higher than ULN of the testing laboratory.
18. Have clinically significant cardiac disease including any of the following: congestive heart failure requiring treatment (New York Heart Association [NYHA] Grade ≥2), left ventricular ejection fraction (LVEF)<50% or local lower limit of normal as determined by multiple gated acquisition (MUGA) scan or echocardiogram (ECHO), or uncontrolled hypertension (refer to European Society of Cardiology and European Society of Hypertension guidelines [Williams et al., 2018]); presence of Common Terminology Criteria for Adverse Events (version 5.0 or later) Grade ≥2 ventricular arrhythmias, atrial fibrillation, bradycardia, or conduction abnormality; unstable angina pectoris or acute myocardial infarction ≤3 months before the first dose of study drug; QTcF >470 msec (males and females). Note: If the QTcF is >470 msec in the first electrocardiogram (ECG), a total of 3 ECGs separated by at least 5 minutes should be performed. If the average of these 3 consecutive results for QTcF is ≤470 msec, the subject meets eligibility in this regard; history of congenital long QT syndrome.
19. Have had a recent (≤3 months prior to the first dose of study drug) transient ischemic attack or stroke.
20. If female, are pregnant or nursing (lactating), where pregnancy is defined as the state of a female after conception and until the termination of gestation, confirmed by a positive human chorionic gonadotrophin urine or blood laboratory test.
21. Have a known allergy/hypersensitivity reaction to any components of the study drug.
22. Have any other concurrent disease or condition that, in the view of the Investigator, would interfere with study participation.

Subjects randomized to the infigratinib group receive hard gelatin capsules for oral administration of infigratinib 125 mg QD (administered as one 100-mg capsule and one 25-mg capsule) using a 3 weeks on (Days 1-21)/1 week off (Days 22-28) dosing schedule.

Subjects randomized to placebo receive placebo matching in appearance the active study drug (infigratinib), which is provided as hard gelatin capsules for oral use and administered once daily on a 3 weeks on (Days 1-21)/1 week off (Days 22-28) dosing schedule.

### Duration of Treatment

Subjects receive treatment for up to 52 weeks.

### Criteria for Evaluation

Efficacy: Assessments consist of computed tomography (CT)/magnetic resonance imaging (MRI) scans performed at baseline within 28 days before start of treatment, every 3 months up to 24 months, at C13D28 or end of treatment (EOT), and annually thereafter or until metastatic recurrence by investigator assessment if local/regional or contralateral invasive recurrence by BICR has already occurred. Cystoscopy and cytology (for UTUC subjects with a bladder) is performed at Screening; 3, 6, 9, and 12 months; at C13D28 or EOT; then every 6 months up to 24 months, and then annually or until metastatic recurrence by BICR or metastatic recurrence by investigator assessment if local/regional or contralateral invasive recurrence by BICR has already occurred. Subjects who come off treatment before the end of the year of treatment for reasons other than recurrence should continue completing efficacy assessments per the Schedule of Assessments (Table 3: Schedule of Assessments: PK Sampling).

QOL: Subject QOL is evaluated at Screening and at every visit through the first 6-month follow-up visit after discontinuation of study drug using the EORTC QLQ-C30 and EQ-5D.

PK: Blood samples are collected Cycle 1 Day 1 predose and 4 hours (±30 min) postdose; on Cycle 1 Day 21 predose and 4 hours (±30 min) postdose; and on Cycle 2 and all subsequent cycles on Day 21 predose and 4 hours (±30 min) postdose. Plasma concentrations of infigratinib and its active metabolites are measured. The pharmacokinetic (PK) parameter of C_{trough} and Cₘₐₓ is also calculated.

Safety: Assessments are collected at Screening and every visit throughout the treatment period (see Table 2) and up to 30-days post-treatment: adverse events (AEs and serious AEs [SAES]), clinical laboratory tests (blood and urine), physical examinations, vital signs, and electrocardiograms (ECGs), LVEF (ECHO or MUGA), ECOG, ophthalmic assessments. Retinal optical coherence tomography (OCT) scan images are sent for BICR. AEs and SAEs are assessed 30 days post-treatment.

### Statistical Methods

Sample Size: Approximately 218 subjects will be initially randomly assigned to treatment in this study in a double-blind fashion. The sample size can be increased up to a total of 328 subjects based on interim analysis result using an adaptive design promising zone approach. The study will start with a group sequential design with 1 interim analysis at approximately 35 centrally reviewed DFS events (50% of the initial event goal). A Haybittle-Peto boundary will be used for the efficacy boundary with a fixed one-sided alpha of 0.00005 spent at the interim analysis for centrally reviewed DFS, and the rest of the alpha (one-sided alpha=0.025) spent at the primary centrally reviewed DFS analysis. Though an efficacy boundary is specified for the interim centrally reviewed DFS analysis, the trial will not stop at the interim analysis if the efficacy boundary is crossed. A Lan DeMets spending function approximating O'Brien-Fleming boundaries will be used for the non-binding futility boundary. Assuming disease will recur in 46% of subjects in the first 2 years and a 5% yearly recurrence rate in the third year and beyond for the placebo group, the required sample size with initial group sequential design is approximately 218 subjects to reach 70 centrally reviewed DFS events. This is assuming with 3-year uniform enrollment, 1-year follow-up, 10% yearly drop-out rate, and a hazard ratio (HR) of 0.5. The sample size will provide approximately 80% power to detect a difference in DFS assuming an HR of 0.5, based on a log-rank test controlling type I error at one-sided 0.025.

At the interim analysis, the study uses an adaptive design promising zone approach to adjust sample size and event goal as needed. The details of the sample size adaptation method will be prespecified in the adaptation plan. If no sample size adaption is needed at the interim analysis, the study is projected to reach the planned number of centrally reviewed DFS events (70) 4 years from the randomization of the first subject. If a sample size increase is deemed necessary based on the interim result and the promising zone approach, the sample size/event goal will be increased by maximum of 50% (328/105). If sample size is increased and event goal is adjusted, then the subsequent analyses will be adjusted accordingly time-wise when the adjusted event goal is reached, and the boundary to test centrally reviewed DFS when the adjusted event goal is reached will be based on the original boundary from the initial group sequential design.

Efficacy Analyses: The primary efficacy analysis is conducted on the intent-to-treat (ITT) population, which includes all subjects who are randomized. Subjects are analyzed according to the treatment arm to which they are randomized.

For the primary efficacy endpoint, CHW statistics based on stratified log-rank test (using randomization stratification factors except disease type [UTUC or UBC]) will be used to control type I error in case of sample size increase at the interim analysis. Conventional stratified log-rank test will be used for the inference on centrally reviewed DFS if sample size is not adjusted at interim. Repeated confidence interval will be provided for the estimated HR based on stratified Cox proportional hazard model.

For the secondary efficacy endpoints DFS (including intraluminal low-risk recurrence), MFS, and OS, a fixed sequence testing procedure will be followed to control the family-wise type I error at a level of one-sided 0.025.

DFS including intraluminal low-risk recurrence will be tested first if the test on centrally reviewed DFS is significant, followed by the test on MFS if both DFS and DFS including intraluminal low-risk recurrence are significant. OS will be tested finally if DFS, DFS including intraluminal low-risk recurrence, and MFS are all significant.

Interim Analysis: One formal interim analysis of DFS is performed when a total of 35 DFS events have occurred.

At the interim analyses, the study will not be stopped for efficacy if the efficacy boundary for centrally reviewed DFS is crossed.

The study may be stopped due to futility at the interim DFS analysis if the futility boundary for testing centrally reviewed DFS is crossed. The futility stopping boundary is non-binding to allow for additional considerations.

If a sample size increase is deemed necessary based on the interim result on DFS using the promising zone approach, the sample size/event goal is increased by a maximum of 50% (328/105). The details of the sample size adaptation method are pre-specified in a separate adaptation plan.

### Example 5: A Marker Lesion Study of Oral Infigratinib (BGJ398) in Patients with Non-Muscle Invasive Bladder Cancer with FGFR3 Genomic Alterations

### Study Design

Patients with clinical high-grade non-invasive papillary urothelial carcinoma that had recurred following prior treatment with intravesical Bacillus Calmette-Guerin (BCG) were eligible for the study.

FGFR3 alteration status of the patients was determined via testing of pre-treatment or archival tumor tissue.

BGJ398 was administered orally to eligible patients at a dose of 125 mg PO using a 3 week on, 1 week off dosing schedule (1 cycle). Response was determined after 2 cycles of treatment (at 7 weeks) via cystoscopy and urine cytology. Patients with a complete response were given the option to continue therapy for an additional 11 months.

### Results

Four patients were enrolled in the trial. Two of the patients were determined to have the FGFR3 S249C mutation, one patient had the FGFR3 K650E mutation, and one patient had a FGFR3-TACC3 fusion.

Three of patients showed a complete response at the 7-week evaluation timepoint. The other patient exhibited a smaller, necrotic-appearing lesion after discontinuing the treatment at 4 weeks.

Clinically significant toxicities included eye disorders, skin and nail disorders, and elevations in liver function tests (LFTs). Two of the patients required dose reductions for toxicities. Two of the patients discontinued treatment before the 7-week evaluation, one due to skin toxicity and the other due to hepatotoxicity. The other two patients continued treatment after a complete response at 7 weeks, but eventually discontinued treatment after 3 and 4 cycles of treatment (after 11 and 16 weeks) for vision/skin toxicities and nail infection/mucositis, respectively.

TABLE 4
Study Results

| | | | | | | Reasons for |
|---|---|---|---|---|---|---|
| | FGFR3 | Treatment | | Disease-Free | Dose | Treatment |
| Patient | Alteration | Duration | Response | Interval | Reductions | Discontinuation |
| 1 | FGFR3- | 11 weeks | Complete | 6 months | C2, C3: 100 mg | Nail pain, mild |
| | TACC3 | | Response | (LG Ta) | | blurry vision |
| | fusion | | | | | |
| 2 | FGFR3 | 6 weeks | Complete | 12 months | None | LFT elevation |
| | K650E | | Response | (LG Ta) | | |
| 3 | FGFR3 | 4 weeks | Indeterminate | 6+ months | None | Ectopic |
| | S249C | | | (ongoing) | | mineralization |
| 4 | FGFR3 | 16 weeks | Complete | 3+ months | C3: 100 mg | Nail infection, |
| | S249C | | Response | (ongoing) | C4: 75 mg | mucositis |

### Example 6: A Study of the Tolerability and Activity of Neoadjuvant Infigratinib (BGJ398) in Upper Tract Urothelial Carcinoma

### Objectives of Study

Primary objective: evaluate the tolerability of infigratinib in patients with low-grade and high-grade platinum ineligible upper tract urothelial carcinoma (UTUC).

Secondary objectives include: assessment of tolerability of infigratinib in those with a Glomerular filtration rate (GFR) of 30-44; evaluation of the objective response rate (complete response (CR) and partial response (PR)) of infigratinib after 2 cycles in UTUC with and without FGFR3 alterations; correlation of tumor tissue FGFR3 alteration (presence/absence, alteration type, and clonal status) with response and occurrence/severity of adverse events (AEs) such as hyperphosphatemia; upper tract, bladder and local/distant recurrence within 12 months; renal function is evaluated pre-treatment and after two treatments.

Objectives include: evaluation of intra-tumor heterogeneity, gene expression profiles, and changes in tumor microenvironment using single cell RNA sequencing (scRNA-seq) and mass cytometry (CyTOF) pre and post treatment to identify potential mechanisms of response and/or resistance, and correlation with the occurrence/severity of AEs; urinary/upper tract washing FGFR3 alterations as potential biomarker for detection and response; cell-free DNA (cfDNA) for detection of FGFR3 alterations and as a predictor of response.

Primary endpoint: the proportion of patients unable to complete 2 cycles of treatment due to excessive toxicity.

Secondary endpoints include the percentage of patients achieving objective response (CR or PR) after 2 cycles of infigratinib based on pathologic evaluation. Tumor mapping is performed from the endoscopic evaluation and used to compare to pathologic findings in order to determine responses.

Other possible endpoints include:

(1) Tumor Studies: scRNA-seq is performed on fresh frozen tumors using a 10× Genomics platform. Tumor cell heterogeneity, FGFR3 gene expression, and tumor microenvironment is profiled. All bioinformatics data analysis is performed in the Computational Biology Laboratory, Department of Genomic Medicine; tissue microarray (TMA) is constructed from FFPE tissue (biopsy and final pathologic specimen) and undergo interrogation for immunologic studies using CyTOF. For patients with a complete response without residual tumor, the bed of the largest pretreatment tumor (based on tumor map) is used for immune studies; tissue prioritization: use of biopsy and pathologic tumor tissue is prioritized in the following order and sources: 1. Mutational analysis (FFPE), 2. RNAseq (fresh/frozen), 3. TMA (FFPE).
(2) Urinary Biomarkers: voided urine is collected preferentially but substituted with selective upper tract washings when voided urine is not available or insufficient. Urine and blood is collected at 3 time points (pretreatment, after completion of infigratinib treatment/preoperatively, and 5 weeks+/−2 weeks postoperatively). Urine processing follows established standard operating procedures. Samples are stored at −80° C. and then sent to the Fox Chase Cancer Center for further analyses (Dr. Phil Abbosh laboratory, with whom we have an existent collaboration and MTA). DNA is isolated from the urine sample and then checked for quality (typically yielding several micrograms of high molecular weight DNA). DNA is also isolated from peripheral blood mononuclear cells (PBMCs) prior to initiation of therapy to use as a germline reference sample. DNA from the germline and pre/post-treatment/post-op time points are subjected next generation sequencing using the HaloPlexHS platform with a targeted depth of 1000× covering 54 well characterized cancer genes. These genes are enriched in patients with urothelial carcinoma (including FGFR3). HaloPlexHS uses pre-amplification single molecule tags to filter taq errors occurring during PCR, thus greatly enhancing the power to detect rare alleles. In preliminary experiments, this approach was validated to be highly sensitive and accurate, detecting >60% of tumor tissue mutations in the urine and identifying additional mutations in the urine that were not seen in tissue. Urine is characterized for FGFR3 hotspots or other missense variants and their variant allele frequency is tracked in longitudinal samples. The presence of point mutations in the pre-treatment urine is correlated with pathological response as an a priori predictive biomarker. Separately, clearance of all pre-treatment mutations after treatment is correlated with infigratinib and after surgery with pathologic response as a post hoc biomarker. Correlation is determined using Fishers exact test for both analyses.
(3) Cell-Free DNA (cfDNA): analysis of the association of cfDNA with response, blood is collected at enrollment and post-treatment/preoperatively (30 mL at each time point). These samples are processed and stored until tumor studies are completed and the results are available. Of all patients identified as having tumor FGFR3 alterations, 5 are randomly selected to have their baseline cfDNA assayed; if 3 or more are found to have detectable FGFR3 alterations, then up to 5 more patient baseline samples will be run. Those found to have detectable baseline FGFR3 alterations in their cfDNA have their second time point assayed. These results are then correlated to disease burden, pathologic findings, disease grade, stage, objective response, and immune correlates. For cfDNA, the availability of a 70-Gene Liquid Biopsy Panel (LBP-70) is leveraged. The validated next generation sequencing (NGS)-based panel is run in the MD Anderson Department of Pathology and Laboratory Medicine. Peripheral blood is collected into Streck tubes designed to reduce admixture of circulating cfDNA with cellular DNA from blood cells during transport. The NGS-based panel is designed to detect single nucleotide variants (SNVs) and small insertion-deletions (Indels) in all 70 genes included in the panel. In addition, amplifications (copy number variants; CNVs) and fusions (translocations) involving selected genes can also be detected. Specifically in regard to this study, the panel is able to detect mutations/indels, amplifications, and fusions of FGFR3. The comprehensive liquid biopsy test utilizes molecular barcode technology and sophisticated error detection algorithms to allow a sensitive and accurate detection of low level mutations.

### Study Design

Patients undergoing planned surgical resection with nephroureterectomy or ureterectomy having adequate biopsy tissue for biomarker studies and are not candidates for neoadjuvant chemotherapy (low-grade disease, or high-grade platinum-ineligible) take infigratinib for 2 cycles (1 cycle=3 weeks on, 1 week off) and then undergo nephroureterectomy or ureterectomy. Patients are continually monitored for safety and tolerability. Redundant tumor and normal tissues are harvested for biomarker studies at the time of surgical resection by a dedicated study GU pathologist. Pre-treatment biopsy is evaluated for FGFR3 alterations, and surgical specimen tissue is evaluated for objective response to treatment (complete response and partial response, in comparison to pre-treatment endoscopic assessment using tumor mapping); the presence of FGFR3 alterations is then correlated with objective response.

Dosage: all patients receive infigratinib 125 mg orally once daily (QD) using a 3 weeks on, 1 week off schedule for each 28-day treatment cycle, which will be repeated for a total of 2 cycles. Surgery is performed during week 8-9.

Number of subjects: 20 subjects are initially planned for study participation.

### Inclusion Criteria

Eligible subjects meet all of the following criteria:
1. Subjects have low grade UTUC undergoing nephroureterectomy or ureterectomy, or high grade UTUC and are not eligible for cis-platin neoadjuvant chemotherapy either due to medical comorbidities (e.g., cardiac dysfunction, hearing loss, GFR <50), or based on <49% risk prediction of non-organ confined disease by clinical nomogram (Petros F. et al., Urol. Oncol., 2018).
2. Subjects have adequate biopsy tissue available for mutational analysis, as determined by the study pathologist, prior to enrollment.
3. Subjects have an Eastern Cooperative Oncology Group (ECOG) performance status of 0-2.
4. Subjects have recovered from AEs of previous systemic anti-cancer therapies to baseline or Grade 1, except for alopecia.

### Exclusion Criteria

Eligible subjects do not meet any of the following criteria:
1. Subjects have a history of another primary malignancy within 3 years except: a. adequately treated in situ carcinoma of the cervix, or non-melanoma carcinoma of the skin, b. any other untreated cancer deemed by the treating physician to be at low risk for progression during the study period (e.g., low or intermediate risk prostate cancer), c. a curatively treated malignancy that is not expected to have recurrence or require treatment during the course of the study.
2. Subjects have uncontrolled bladder cancer. Patients with bladder cancer must have a bladder cleared of disease by transurethral resection prior to initiating treatment and must not be at need for systemic therapy or cystectomy.
3. Subjects have current evidence of corneal or retinal disorder/keratopathy including, but not limited to, bullous/band keratopathy, corneal abrasion, inflammation/ulceration, and keratoconjunctivitis, confirmed by ophthalmologic examination. Subjects with asymptomatic ophthalmologic conditions assessed by the investigator to pose minimal risk for study participation may be enrolled in the study.
4. Subjects have a history and/or current evidence of extensive tissue calcification including, but not limited to, the soft tissue, kidneys, intestine, myocardium and lung with the exception of calcified lymph nodes, minor pulmonary parenchymal calcifications, and asymptomatic coronary calcification.
5. Subjects have impairment of gastrointestinal (GI) function or GI disease that may significantly alter the absorption of oral infigratinib (e.g., ulcerative diseases, uncontrolled nausea, vomiting, diarrhea, malabsorption syndrome, small bowel resection).
6. Subjects have current evidence of endocrine alterations of calcium/phosphate homeostasis, e.g., parathyroid disorders, history of parathyroidectomy, tumor lysis, tumoral calcinosis, etc.
7. Subjects are currently receiving treatment with agents that are known strong inducers or inhibitors of CYP3A4 and medications which increase serum phosphorus and/or calcium concentration. Subjects are not permitted to receive enzyme-inducing anti-epileptic drugs, including carbamazepine, phenytoin, phenobarbital, and primidone.
8. Subjects have consumed grapefruit, grapefruit juice, grapefruit hybrids, pomegranates, star fruits, pomelos, Seville oranges or products containing juice of these fruits within 7 days prior to first dose of study drug.
9. Subjects have used medications known to prolong the QT interval and/or are associated with a risk of Torsades de Pointes (TdP) 7 days prior to first dose of study drug.
10. Subjects have used amiodarone within 90 days prior to first dose of study drug.
11. Subjects are currently using therapeutic doses of warfarin sodium or any other coumadin-derivative anticoagulants or using direct thrombin inhibitors (e.g., argatroban) or Factor Xa inhibitors (e.g., rivaroxaban) that are primarily metabolized by CYP3A4. Heparin and/or low molecular weight heparins or direct thrombin inhibitors and/or Factor Xa inhibitors that are not metabolized by CYP3A4 (e.g., dabigatran, edoxaban) are allowed.
12. Subjects have insufficient bone marrow function:
   (a) Absolute neutrophil count (ANC)<1,000/mm³ (1.0×10⁹/L)
   (b) Platelets <100,000/mm³ (75×10⁹/L)
   (c) Hemoglobin <9.0 g/dL
13. Subjects have insufficient hepatic and renal function:
   (a) Total bilirubin >1.5× upper limit of normal (ULN) (unless documented Gilbert's syndrome, and then only by approval by study medical monitor)
   (b) Aspartate aminotransferase/serum glutamic-oxaloacetic transaminase (AST/SGOT) and alanine aminotransferase/serum glutamic-pyruvic transaminase (ALT/SGPT) >2.5×ULN (AST and ALT >5×ULN in the presence of liver involvement of cholangiocarcinoma)
   (c) Calculated or measured creatinine clearance of <30 mL/min
14. Subjects have amylase or lipase >2.0×ULN.
15. Subjects have abnormal calcium-phosphate homeostasis:
   (a) Inorganic phosphorus outside of local normal limits
   (b) Total corrected serum calcium outside of local normal limits
16. Subjects have clinically significant cardiac disease including any of the following:
   (a) Congestive heart failure requiring treatment (New York Heart Association Grade ≥2), left ventricular ejection fraction (LVEF)<50% or local lower limit of normal as determined by echocardiogram (ECHO), or uncontrolled hypertension (refer to the European Society of Cardiology and European Society of Hypertension guidelines)
   (b) Presence of Common Terminology Criteria for Adverse Events (CTCAE) v5.0 Grade ≥2 ventricular arrhythmias, atrial fibrillation, bradycardia, or conduction abnormality
   (c) Unstable angina pectoris or acute myocardial infarction ≤3 months prior to first dose of study drug
   (d) QTcF >470 msec (males and females). Note: If the QTcF is >470 msec in the first electrocardiogram (ECG), a total of 3 ECGs separated by at least 5 minutes should be performed. If the average of these 3 consecutive results for QTcF is ≤470 msec, the subject meets eligibility in this regard.
   (e) Known history of congenital long QT syndrome.
17. Subjects have had a recent (≤3 months) transient ischemic attack or stroke.
18. CTCAE (v5.0) Grade ≥2 hearing loss
19. CTCAE (v5.0) Grade ≥2 neuropathy
20. If female, is pregnant or nursing (lactating), where pregnancy is defined as the state of a female after conception and until the termination of gestation, confirmed by a positive human chorionic gonadotrophin urine or blood laboratory test.

Oral infigratinib 125 mg QD is administered to subjects (administered as one 100-mg capsule and one 25-mg capsule) using a “3 weeks on, 1 week off” schedule for each 28-day treatment cycle. Two 28-day cycles using a “3 weeks on, 1 week off” for each of them.

Safety: the safety evaluation is based on AE reporting, laboratory parameters, vital signs, physical examinations, 12-lead ECGs, and ophthalmic assessments. Tolerability is assessed by the incidence of AEs leading to study drug discontinuation.

Efficacy: tumor response will be evaluated by comparing the tumor mapping at the time of endoscopic evaluation prior to treatment with the pathological findings in the surgical specimen.

Primary Objective: the primary objective is to evaluate the safety and tolerability of Infigratinib as neoadjuvant therapy. Up to 20 patients are enrolled in the study. The study estimates the proportion of patients who are not able to complete treatment (discontinuation before completing 2 cycles of treatment) due to excessive toxicity along with the 90% exact confidence interval. Toxicities are tabulated using frequency and percentage by grade and their relations to treatment. Assuming a 30% of discontinuation due to excessive toxicity, the 90% confidence interval would be (13.1%, 46.9%) with a sample size of 20.

Toxicity data is summarized for the whole group of patients and for patients with eGFR≥50 and eGFR [i.e. 30-49] separately. The safety analysis includes all patients who receive at least one dose of infigratinib.

Safety Monitoring: the study monitors the incidence of not completing treatment due to excessive toxicity using this monitoring rule. That is stop the trial early if Prob (Ptox>0.3| data) >0.85. Where Ptox denotes the proportion of patients not being able to complete treatment. The corresponding stopping boundaries are that the trial is stopped early if at any time it is observed that (n of patients not completing treatment due to excessive toxicity/N of patients treated) >=3/5, 4/(6−8), 5/(9−10), 6/(11−13), 7/(14−16), and 8/(17−19). This stopping rule is applied for each cohort of eGFR ≥45 and eGFR 30-44 separately, such that enrollment is halted only for the affected GFR cohort.

Secondary Objective (Efficacy): the secondary efficacy endpoint is objective response after 2 cycle treatment of infigratinib. The objective response rate is estimated along with the 90% confidence interval for the whole cohort of patients, and for patients with and without FGFR3 alterations. Fisher's exact test is used to explore the difference in response between the two cohorts of patients.

Secondary Objective (Other Secondary Analysis and Exploratory Analyses): descriptive statistics is used to summarize quantifications as continuous variables and frequency and percentage along with 95% CI is used to summarize categorical variables. Wilcoxon rank sum test and Fisher's exact test is used to explore the association between objective response and secondary outcomes, e.g. cfDNA, expression of markers, FGFR3 alteration type. The same methods is also used to explore the association of AEs such as hyperphosphatemia with response. Recurrence at 12 months is summarized using proportion and 90% confidence interval as a binary outcome and is estimated using the Kaplan-Meier method as a time to event variable. All analyses are performed for all patients and on patients stratified as having or not having FGFR3 alterations.

Sample Size: 20 participants.

Safety Analyses: full details of the planned statistical analyses are included in the Statistical Analysis Plan.

### Example 7: A Study of the of Infigratinib (BGJ398) in Upper Tract Urothelial Carcinoma Compared to Urothelial Carcinoma of the Bladder and Association with Comprehensive Genomic Profiling/Cell-Free DNA Data

### Objectives

Determine whether there are differences in infigratinib activity in upper tract urothelial carcinoma (UTUC) and urothelial carcinoma of the bladder (UCB), given the distinct biological characteristics of UTUC and UCB.

Determine whether UTUC and UCB differed in their genomic profiles in patients with advanced or metastatic urothelial carcinoma through characterization of tumor tissue and cell-free DNA (cfDNA).

### Study Design

Eligible patients had metastatic urothelial carcinoma with activating FGFR3 mutations/fusions and prior platinum-based chemotherapy, unless contraindicated.

Patients received 125 mg of infigratinib by oral administration, once daily, using a dosing regimen of 3 weeks on/1 week off

Overall response rate (ORR: CR+PR) and disease control rate (DCR; CR+PR+SD) were characterized in UCB and UTUC patients.

Genomic profiling of UCB and UTUC patients was performed with DNA isolated from FFPE tumor tissue and plasma (cfDNA) obtained prior to treatment:
Comprehensive genomic profiling of tumor tissue (Foundation Medicine; Cambridge, Mass.) was used patients to enroll patients with genetic alterations in FGFR3.
Cell-free DNA (cfDNA) obtained from blood prior to treatment was evaluated by next-generation sequencing using a 600-gene panel (Novartis Labs).
The baseline characteristics of the patients that participated in the study are shown in Table 5.

TABLE 5
Baseline Characteristics of Patients

| | UTUC | UCB | Total |
|---|---|---|---|
| Characteristic | (n = 8) | (n = 59) | (n = 67) |
| Age | | | |
| <65 years | 4 (50.0) | 25 (42.4) | 29 (43.3) |
| ≥65 years | 4 (50.0) | 34 (57.6) | 38 (56.7) |
| Visceral disease, n (%) | | | |
| Lung | 5 (62.5) | 36 (61.0) | 41 (61.2) |
| Liver | 2 (25.0) | 23 (39.0) | 25 (37.3) |
| Lymph node metastases, n (%) | | | |
| Yes | 2 (25.0) | 26 (44.1) | 19 (28.4) |
| No | 6 (75.0) | 33 (55.9) | 46 (68.7) |
| Bony metastases, n (%) | | | |
| Yes | 3 (37.5) | 23 (39.0) | 25 (37.3) |
| No | 5 (62.5) | 36 (61.0) | 40 (59.7) |

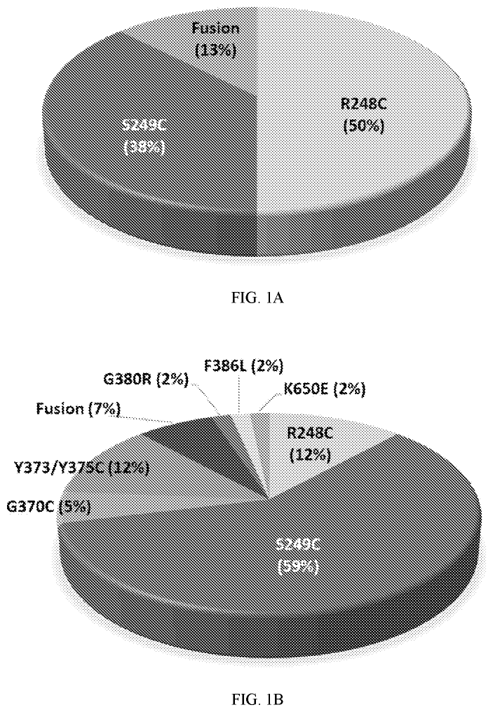

A different frequency of mutations R248C and S249C in the FGFR3 extracellular Ig-like domains was observed in UTUC compared to UCB (FIGS. 1A and 1B, respectively). Mutations outside of the Ig-like domains were observed in UCB but not UTUC.

A summary comparing the efficacy of prior anti-cancer therapies in patients with UTUC or UCB is shown in Table 6.

TABLE 6
Efficacy Summary for Prior Anti-Cancer Therapies

| | UTUC | UCB | Total |
|---|---|---|---|
| | (n = 8) | (n = 59) | (n = 67) |
| Total number of lines of prior | | | |
| therapies, n (%) | | | |
| 0 | 0 | 13 (22.0) | 13 (19.4) |
| 1 | 5 (62.5) | 19 (32.2) | 24 (35.8) |
| ≥2 | 3 (37.5) | 27 (45.7) | 30 (44.8) |
| Total number of prior anticancer | | | |
| regimens, n (%) | | | |
| 0 | 0 | 1 (1.7) | 1 (1.5) |
| 1 | 2 (25.0) | 17 (28.8) | 19 (28.4) |
| ≥2 | 6 (75.0) | 41 (67.8) | 47 (70.1) |
| Best response to prior anticancer | | | |
| regimen, n (%) | | | |
| Complete response (confirmed) | 0 | 1 (1.7) | 1 (1.5) |
| Completed response (unconfirmed) | 0 | 1 (1.7) | 1 (1.5) |
| Partial response | 2 (25.0) | 8 (13.6) | 10 (14.9) |
| Stable disease | 2 (25.0) | 21 (35.6) | 23 (34.3) |
| Progressive disease | 2 (25.0) | 14 (23.7) | 16 (23.9) |
| Missing | 2 (25.0) | 14 (23.7) | 16 (23.9) |

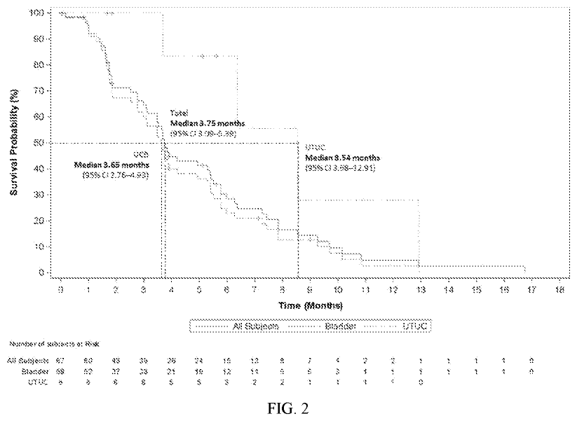

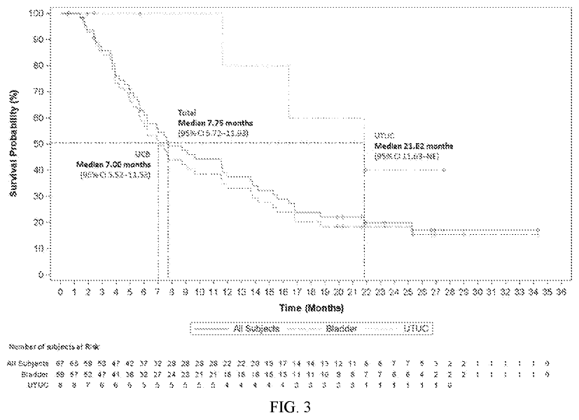

A summary of the study comparing the efficacy of infigratinib in patients with UTUC or UCB is shown in Table 7. FIG. 2 and FIG. 3 are overlays of the progression-free survival and overall survival rates, respectively, for patients treated with infigratinib.

TABLE 7
Efficacy Summary for Infigratinib

| | UTUC | UCB | Total |
|---|---|---|---|
| | (n = 8) | (n = 59) | (n = 67) |
| Response assessment, n (%) | | | |
| Complete response (CR), | 1 (12.5) | 0 | 1 (1.5) |
| confirmed | | | |
| Partial response (PR), | 3 (37.5) | 13 (22.0) | 16 (23.9) |
| confirmed | | | |
| CR/PR, unconfirmed | 1 (12.5) | 10 (16.9) | 11 (16.4) |
| Stable disease (SD) | 4 (50.0) | 22 (37.3) | 26 (38.8) |
| Progressive disease | 0 | 18 (30.5) | 18 (26.9) |
| Unknown/not done | 0 | 6 (10.2) | 6 (9.0) |
| Confirmed objective response | 4 (50.0) | 13 (22.0) | 17 (25.5) |
| (CR or PR), n (%) | | | |
| Censored^{a} | 1 (25.0) | 3 (23.1) | 4 (23.5) |
| 95% CI | 15.7-84.3 | 12.3-34.7 | 15.5-37.5 |
| Best overall response (CR or | 5 (62.5) | 23 (39.0) | 28 (41.8) |
| PR, conf/unconf), n (%) | | | |
| 95% CI | 24.5-91.5 | 26.5-52.6 | 29.8-54.5 |
| Disease control rate | 8 (100.0) | 35 (59.3) | 43 (64.2) |
| (CR/PR or SD), n (%) | | | |
| 95% CI | 63.1-100.0 | 45.7-71.9 | 51.5-75.5 |
| Median duration of | 6.77 | 5.04 | 5.62 |
| response, months | | | |
| Range | 3.32+-11.01 | 2.33+-8.08 | 2.33+-11.01 |

^{a}Patients who have a confirmed objective response without an assessment of disease progression/deaths are included as ‘censored’

Patient 0507_00103 (see Table 7 for UTUC patient with complete response): 62 year old woman with Stage III UTUC (target lesions in the lung and mediastinum and non-target lung nodules at baseline). She started infigratinib 125 mg 3 weeks on/1 week off 29 May 2013 and remains on treatment (50 mg 3 weeks on/1 week off). CR started 19 Aug. 2016 and is continuing.

A summary of the treatment emergent adverse events (TEAEs) observed during the study is shown in Table 8.

TABLE 8
Summary of TEAEs

| | UTUC | | UCB | Total |
|---|---|---|---|---|
| n (%) | (n = 8) | | (n = 59) | (n = 67) |
| Any TEAE | 8 | (100.0) | 58 (98.3) | 66 (98.5) |
| Grade 3/4 | 5 | (62.5) | 41 (69.5) | 46 (68.7) |
| Serious | 1 | (12.5) | 23 (39.0) | 24 (35.8) |
| Treatment-related | 8 | (100.0) | 56 (94.9) | 64 (95.5) |
| Serious treatment-related | 1 | (12.5) | 3 (5.1) | 4 (6.0) |
| Leading to treatment | 2 | (25.0) | 8 (13.6) | 10 (14.9) |
| discontinuation | | | | |
| Leading to dose | 8 | (100.0) | 45 (76.3) | 53 (79.1) |
| interruption/dose adjustment | | | | |

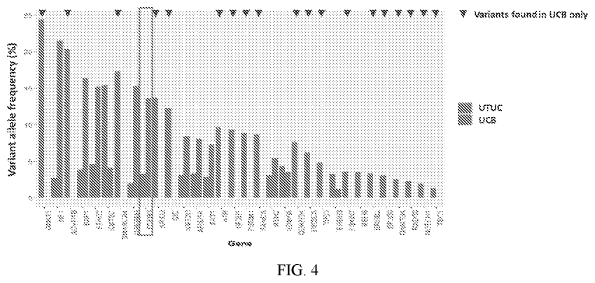

It was observed that across the majority of genes the variant allele frequency (VAF) in cfDNA was higher in UCB than in UTUC (FIG. 4). The higher VAF in cfDNA observed in UCB suggests that UCB patients may have higher disease burden or different mechanisms of metastasis compared with UTUC.

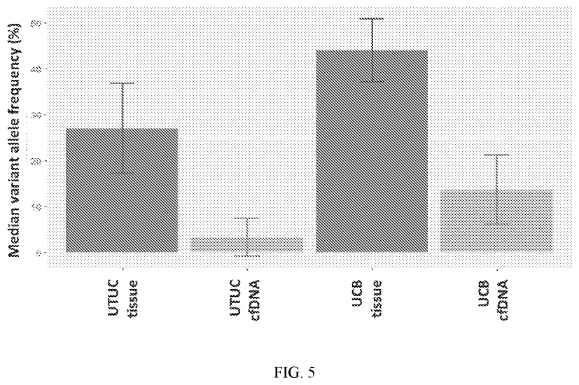

The median VAF for FGFR3 genomic alterations was higher in tumor tissue and cfDNA in UCB patients compared to UTUC patients (FIG. 5).

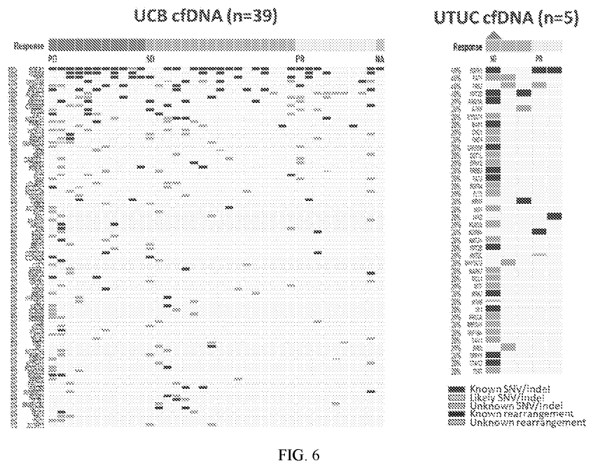

FGFR3 alterations were concordant in 30/38 (79%) of patients with both tumor tissue and cfDNA at screening. A more complex genomic profile with an increased mutational burden was observed in cfDNA from UCB patients compared to UTUC patients (FIG. 6). UTUC patient with high mutation load (▴) is likely deficient in mismatch repair due to frameshift mutation in MSH2.

### INCORPORATION BY REFERENCE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

We claim:

1. A method of treating an upper tract urothelial carcinoma in a patient in need thereof, comprising administering to the patient an effective amount of infigratinib or a pharmaceutically acceptable salt thereof.

2. The method of claim 1, wherein the upper tract urothelial carcinoma is an invasive upper tract urothelial carcinoma.

3. The method of claim 1, wherein the upper tract urothelial carcinoma is a non-invasive upper tract urothelial carcinoma.

4. The method of any one of claims 1-3, wherein the patient is not eligible for treatment with a cisplatin-based chemotherapeutic therapy.

5. The method of any one of claims 1-3, wherein the patient has previously been administered a cisplatin-based chemotherapy but has a residual carcinoma.

6. The method of any one of claims 1-5, wherein administering an effective amount of infigratinib, or a pharmaceutically acceptable salt thereof, occurs following a nephro-ureterectomy or a distal ureterectomy.

7. The method of any one of claims 1-6, wherein administering an effective amount of infigratinib, or a pharmaceutically acceptable salt thereof, to the patient has greater efficacy in treating the upper tract urothelial carcinoma compared to treating urothelial carcinoma of the bladder by administering the effective amount of infigratinib, or a pharmaceutically acceptable salt thereof, to a patient in need thereof.

8. A method of treating a urothelial carcinoma in a patient in need thereof, comprising administering to the patient an effective amount of infigratinib, or a pharmaceutically acceptable salt thereof, wherein the patient has previously had a nephro-ureterectomy, a distal ureterectomy, or a cystectomy.

9. The method of claim 8, wherein the urothelial carcinoma is an invasive upper tract urothelial carcinoma or urothelial carcinoma of the bladder.

10. The method of claim 8, wherein the urothelial carcinoma is a non-invasive upper tract urothelial carcinoma or urothelial carcinoma of the bladder.

11. The method of claim 8 or 9, wherein the patient is not eligible for treatment with a cisplatin-based chemotherapeutic therapy.

12. The method of claim 8 or 9, wherein the patient has previously been administered a cisplatin-based chemotherapy but has a residual carcinoma.

13. The method of any one of claims 1-12, wherein administering an effective amount of infigratinib, or a pharmaceutically acceptable salt thereof, comprises administering orally about 125 mg of infigratinib, or a pharmaceutically acceptable salt thereof, once daily.

14. The method of any one of claims 1-13, wherein administering an effective amount of infigratinib, or a pharmaceutically acceptable salt thereof, comprises a 28-day cycle, wherein about 125 mg of infigratinib, or a pharmaceutically acceptable salt thereof, is administered orally once daily to the patient for 3 consecutive weeks, and no infigratinib is administered for 1 week.

15. The method of claim 13 or 14, wherein the about 125 mg of infigratinib, or a pharmaceutically acceptable salt thereof, is provided as a 100 mg unit dose and a 25 mg unit dose.

16. The method of claim 13 or 14, wherein the about 125 mg of infigratinib, or a pharmaceutically acceptable salt thereof, is provided as a unit dose.

17. The method of any one of claims 1-12, wherein the effective amount of infigratinib or a pharmaceutically acceptable salt thereof is administered to the patient via local administration.

18. The method of any one of claims 1-16, wherein the urothelial carcinoma is histologically or cytologically confirmed.

19. The method of any one of claims 1-18, wherein the urothelial carcinoma has a FGFR3 mutation, gene rearrangement or gene fusion.

20. The method of any one of claims 1-15, wherein the urothelial carcinoma has a FGFR3 mutation.

21. The method of claim 20, wherein the FGFR3 mutation is selected from the group consisting of FGFR3 R248C, FGFR3 S249C, FGFR3 G372C, FGFR3 A393E, FGFR3 Y375C, FGFR3 K652M/T, FGFR3 K652E/Q, and combinations thereof.

22. A method for treating non-muscle invasive bladder cancer in a patient in need thereof, comprising:
administering an effective amount of infigratinib, or a pharmaceutically acceptable salt thereof, wherein the patient has reoccurrence of the non-muscle invasive bladder cancer after previous administration of another therapy.

23. The method of claim 22, wherein the previous administration of another therapy is a therapy for non-muscle invasive bladder cancer.

24. The method of claim 22 or 23, wherein the previous administration of another therapy is an administration of an immunotherapeutic agent.

25. The method of claim 24, wherein the previous administration of an immunotherapeutic agent is a bacillus Calmette-Guerin-containing regimen.

26. The method of any one of claims 22-25, wherein the non-muscle invasive bladder cancer has a FGFR3 mutation, gene rearrangement or gene fusion.

27. The method of any one of claims 22-26, wherein the non-muscle invasive bladder cancer has a FGFR3 mutation.

28. The method of claim 27, wherein the FGFR3 mutation is selected from the group consisting of FGFR3 K650E, FGFR3 S249C, FGFR3 R248C, FGFR3 Y375C, FGFR3 G372C, FGFR3 S373C, FGFR3 A393E, FGFR3 A371A, FGFR3 I378C, FGFR3 L379L, FGFR3 G382R, and combinations thereof.

29. The method of any one of claims 22-26, wherein the non-muscle invasive bladder cancer has a FGFR3 gene fusion.

30. The method of claim 29, wherein the FGFR3 gene fusion comprises the FGFR3 gene fusion partner TACC3.

31. The method of any one of claims 22-30, wherein administering an effective amount of infigratinib, or a pharmaceutically acceptable salt thereof, comprises administering about 125 mg of infigratinib, or a pharmaceutically acceptable salt thereof, once daily.

32. The method of any one of claims 22-31, wherein administering an effective amount of infigratinib, or a pharmaceutically acceptable salt thereof comprises a 28-day cycle, wherein about 125 mg of infigratinib, or a pharmaceutically acceptable salt thereof, is administered once daily to the patient for 3 consecutive weeks, and no infigratinib is administered for 1 week.

33. The method of claim 31 or 32, wherein the about 125 mg of infigratinib or a pharmaceutically acceptable salt thereof is provided as a 100 mg unit dose and a 25 mg unit dose.

34. The method of claim 31 or 32, wherein the about 125 mg of infigratinib or a pharmaceutically acceptable salt thereof is provided as a unit dose.

35. The method of any one of claims 31-34, wherein the about 125 mg of infigratinib or a pharmaceutically acceptable salt thereof is administered orally to the patient.

36. The method of any one of claims 22-30, wherein the effective amount of infigratinib or a pharmaceutically acceptable salt thereof is administered to the patient via local administration.

37. The method of claim 36, wherein the effective amount of infigratinib or a pharmaceutically acceptable salt thereof is administered to the patient intravesically.

38. The method of claim 36 or 37, wherein the effective amount of infigratinib or a pharmaceutically acceptable salt thereof is delivered via insertion of a controlled release, implantable device into the patient's bladder.

39. The method of claim 36 or 37, wherein the effective amount of infigratinib or a pharmaceutically acceptable salt thereof is delivered via insertion of a controlled release, implantable device into the patient's ureter.

40. The method of claim 36 or 37, wherein the effective amount of infigratinib or a pharmaceutically acceptable salt thereof is delivered via insertion of a controlled release, implantable device into the patient's renal pelvis.

41. The method of any one of claims 38-40, wherein the controlled release, implantable device is a dual-lumen silicon tube comprising a superelastic wireform.

42. The method of any one of claims 38-40, wherein the controlled release, implantable device is a gel.

43. The method of any one of claims 22-42, the method further comprising administering an effective amount of a second therapeutic agent to the patient.

44. The method of claim 43, wherein the effective amount of the second therapeutic agent is administered to the patient via local administration.

45. The method of claim 43 or 44, wherein the effective amount of the second therapeutic agent is administered to the patient intravesically.

46. The method of any one of claims 43-45, wherein the second therapeutic agent is gemcitabine or a pharmaceutically acceptable salt thereof.

47. A method of treating an upper tract urothelial carcinoma in a patient in need thereof, comprising administering to the patient an effective amount of infigratinib or a pharmaceutically acceptable salt thereof, wherein the effective amount of infigratinib or a pharmaceutically acceptable salt thereof is administered as a neoadjuvant therapy.

48. The method of claim 47, wherein the upper tract urothelial carcinoma has a FGFR3 mutation, gene rearrangement or gene fusion.

49. A method of treating a patient in need thereof having an upper tract urothelial carcinoma with at least one FGFR3 mutation, gene rearrangement or gene fusion, the method comprising:
(i) obtaining a sample from the patient;
(ii) analyzing the sample for the presence of the at least one FGFR3 mutation, gene rearrangement or gene fusion; and
(iii) administering to the patient an effective amount of infigratinib or a pharmaceutically acceptable salt thereof,
wherein the effective amount of infigratinib or a pharmaceutically acceptable salt thereof is administered as a neoadjuvant therapy.

50. The method of claim 48 or 49, wherein the urothelial carcinoma has a FGFR3 mutation.

51. The method of claim 50, wherein the FGFR3 mutation is selected from the group consisting of FGFR3 R248C, FGFR3 S249C, FGFR3 G372C, FGFR3 A393E, FGFR3 Y375C, FGFR3 K652M/T, FGFR3 K652E/Q, and combinations thereof.

52. The method of any one of claims 47-51, wherein the upper tract urothelial carcinoma is a low-grade upper tract urothelial carcinoma.

53. The method of claim 47-51, wherein the upper tract urothelial carcinoma is a high-grade upper tract urothelial carcinoma.

54. The method of any one of claims 47-53, wherein the patient is not eligible for treatment with a cisplatin-based neoadjuvant chemotherapeutic therapy.

55. The method of any one of claims 47-54, wherein administering an effective amount of infigratinib, or a pharmaceutically acceptable salt thereof, comprises administering orally about 125 mg of infigratinib, or a pharmaceutically acceptable salt thereof, once daily.

56. The method of any one of claims 47-55, wherein administering an effective amount of infigratinib, or a pharmaceutically acceptable salt thereof comprises a 28-day cycle, wherein about 125 mg of infigratinib, or a pharmaceutically acceptable salt thereof, is administered orally once daily to the patient for 3 consecutive weeks, and no infigratinib is administered for 1 week.

57. The method of claim 56, wherein the effective amount of infigratinib, or a pharmaceutically acceptable salt thereof is administered to the patient for two consecutive 28-days cycles.

58. The method of any one of claims 55-57, wherein the about 125 mg of infigratinib or a pharmaceutically acceptable salt thereof is provided as a 100 mg unit dose and a 25 mg unit dose.

59. The method of any one of claims 55-57, wherein the about 125 mg of infigratinib or a pharmaceutically acceptable salt thereof is provided as a unit dose.

60. The method of any one of claims 49-54, wherein the effective amount of infigratinib or a pharmaceutically acceptable salt thereof is administered to the patient via local administration.

61. The method of any one of claims 47-59, further comprising the patient undergoing a nephro-ureterectomy or a ureterectomy within 8 weeks of commencing the neoadjuvant therapy.

62. A method of identifying a patient for treatment of an upper tract urothelial carcinoma with an effective amount of infigratinib or a pharmaceutically acceptable salt thereof, comprising:
testing a sample obtained from the patient after administration of the effective amount of infigratinib or a pharmaceutically acceptable salt thereof to measure gene expression of at least one FGFR3 biomarker,
wherein detection of an alteration in the level of expression of the at least one FGFR3 biomarker compared to a baseline gene expression measurement is indicative of the candidacy of the patient for treatment, and
wherein the baseline gene expression measurement is the gene expression measured in the patient prior to administration of the effective amount of infigratinib or a pharmaceutically acceptable salt thereof.

63. A method for monitoring the response of a patient to treatment by an effective amount of infigratinib or a pharmaceutically acceptable salt thereof for an upper tract urothelial carcinoma, comprising:
testing a sample obtained from the patient after administration of the effective amount of infigratinib or a pharmaceutically acceptable salt thereof to measure gene expression of at least one FGFR3 biomarker,
wherein detection of an alteration in the level of expression of the at least one FGFR3 biomarker compared to a baseline gene expression measurement is indicative of the response of the patient to the treatment, and
wherein the baseline gene expression measurement is the gene expression measured in the patient prior to administration of the effective amount of infigratinib or a pharmaceutically acceptable salt thereof.

64. A method of identifying a patient for treatment of an upper tract urothelial carcinoma with an effective amount of infigratinib or a pharmaceutically acceptable salt thereof, comprising:
testing a sample obtained from the patient after administration of the effective amount of infigratinib or a pharmaceutically acceptable salt thereof to measure the allele frequency of at least one FGFR3 biomarker in the patient's cell-free DNA (cfDNA),
wherein detection of the at least one FGFR3 biomarker at a lower variant allele frequency in the patient's cfDNA compared to a baseline allele frequency of the at least one FGFR3 biomarker is indicative of the candidacy of the patient for treatment, and
wherein the baseline allele frequency measurement is the allele frequency measured in the patient's cfDNA prior to administration of the effective amount of infigratinib or a pharmaceutically acceptable salt thereof.

65. A method for monitoring the response of a patient to treatment by an effective amount of infigratinib or a pharmaceutically acceptable salt thereof for an upper tract urothelial carcinoma, comprising:
testing a sample obtained from the patient after administration of the effective amount of infigratinib or a pharmaceutically acceptable salt thereof to measure the allele frequency of at least one FGFR3 biomarker in the patient's cell-free DNA (cfDNA),
wherein detection of the at least one FGFR3 biomarker at a lower variant allele frequency in the patient's cfDNA compared to a baseline allele frequency of the at least one FGFR3 biomarker is indicative of the response of the patient to the treatment, and
wherein the baseline allele frequency measurement is the allele frequency measured in the patient's cfDNA prior to administration of the effective amount of infigratinib or a pharmaceutically acceptable salt thereof.
